SEQUENCE LISTING (15 sequences)

SEQ ID NO: 1 - RNA, 20 nt, synthetic construct
  source 1..20: mol_type = other RNA
  ncRNA 1..20: ncRNA_class = guide_RNA
gctactctctctttctggcc

SEQ ID NO: 2 - protein, 9 aa, synthetic construct
VMAPRTLLL

SEQ ID NO: 3 - protein, 8 aa, synthetic construct
VMAPRTLL

SEQ ID NO: 4 - protein, 9 aa, synthetic construct
VMAPRTLFL

SEQ ID NO: 5 - protein, 9 aa, synthetic construct
VMAPRTLVL

SEQ ID NO: 6 - protein, 9 aa, synthetic construct
VMAPRTLIL

SEQ ID NO: 7 - protein, 9 aa, synthetic construct
IMAPRTLVL

SEQ ID NO: 8 - protein, 9 aa, synthetic construct
VMPPRTLLL

SEQ ID NO: 9 - protein, 9 aa, synthetic construct
VMAPRTVLL

SEQ ID NO: 10 - protein, 9 aa, synthetic construct
VTAPRTLLL

SEQ ID NO: 11 - protein, 9 aa, synthetic construct
VTAPRTVLL

SEQ ID NO: 12 - protein, 9 aa, synthetic construct
VMAPRTLTL

SEQ ID NO: 13 - protein, 9 aa, synthetic construct
VMAPRALLL

SEQ ID NO: 14 - protein, 16 aa, synthetic construct
GSSSGSSSGSSSGSSS

SEQ ID NO: 15 - protein, 27 aa, synthetic construct
VMAPRTLFLVMAPRTLFLVMAPRTLFL

(19) United States
(12) Patent Application Publication
(10) Pub. No.: US 2025/0177453 A1
(43) Pub. Date: Jun. 5, 2025

(54) COMPOSITIONS AND METHODS FOR ENHANCING IMMUNE TOLERANCE

(71) Applicant: President and Fellows of Harvard College, Cambridge, MA (US)

(72) Inventors: Douglas A. Melton, Cambridge, MA (US); Dario Gerace, Cambridge, MA (US); Quan Zhou, Cambridge, MA (US)

(21) Appl. No.: 18/942,086

(22) Filed: Nov. 8, 2024

Related U.S. Application Data
(63) Continuation of application No. PCT/US2023/021780, filed on May 10, 2023.
(60) Provisional application No. 63/340,453, filed on May 10, 2022.

(51) Int. Cl. A61K 35/39; A61K 40/10; A61K 40/22; A61K 40/42; A61P 37/06; C07K 14/495; C07K 14/55; C12N 5/071

(52) U.S. Cl. CPC A61K 35/39; A61K 40/10; A61K 40/22; A61K 40/4229; A61K 40/4234; A61P 37/06; C07K 14/495; C07K 14/55; C12N 5/0676; C12N 2506/02; C12N 2510/00

(57) ABSTRACT

Disclosed herein are methods of producing a hypoimmune cell and methods of mediating localized immune tolerance.

### RELATED APPLICATION(S)

This application is a continuation of International Application No. PCT/US2023/021780, filed May 10, 2023, which claims the benefit of U.S. Provisional Application No. 63/340,453, filed on May 10, 2022. The entire teachings of the above applications are incorporated herein by reference.

### STATEMENT REGARDING SEQUENCE LISTING

The Sequence Listing associated with this application is provided in XML format in lieu of a paper copy and is hereby incorporated by reference into the specification. The name of the .xml file containing the Sequence Listing is HRVY-205-WO1.xml. The text file is 14,515 bytes, was created on Aug. 8, 2023, and is being submitted electronically via Patent Center.

### BACKGROUND OF THE INVENTION

Type 1 diabetes (T1D) is an autoimmune disease that results in the destruction of the insulin-producing beta cells of the pancreas. Cadaveric whole pancreas or islet transplantation are successful treatments for T1D; however, they are hampered by the limited number of donors and the requirement for lifelong immunosuppression (Shapiro et al., 2006). To address the shortage of islet material, several protocols have been developed to steer the in vitro differentiation of human induced pluripotent stem cells (iPSCs) into functional SC-islets that include glucose-responsive beta cells (D'Amour et al., 2006; Millman et al., 2016; Nair et al., 2019; Pagliuca et al., 2014; Rezania et al., 2014; Russ et al., 2015; Veres et al., 2019).

Current strategies to protect allografted islet cells include encapsulation (Alagpulinsa et al., 2019; Bochenek et al., 2018), modifying the patient's immune system by co-administration of biologicals such as low dose IL-2 and anti-CD3 (Tepluzimab) (Hartemann et al., 2013; Herold et al., 2019), and/or genetically modifying the SC-islets. Since the main contributors of immune recognition and rejection are the human leukocyte antigens (HLAs), targeting the HLAs has been performed in iPSCs to reduce or eliminate the immune response against foreign cells (Castro-Gutierrez et al., 2021; Deuse et al., 2019; Gornalusse et al., 2017; Han et al., 2019; Harding et al., 2019; Riolobos et al., 2013; Xu et al., 2019; Yoshihara et al., 2020). To date, the engineering strategies demonstrating some immune-protection of SC-islet cells have employed lentiviral over-expression of PD-L1 and the selective retention of a single HLA-A2 allele in HLA-B/C deficient cells (Parent et al., 2021; Yoshihara et al., 2020). However, lentiviral transgene over-expression is limited by transgene silencing (Herbst et al., 2012; Wen et al., 2021), and the retention of a single HLA-A2 allele on SC-islet cells may result in recurrent autoimmune rejection mediated by antigen-specific and tissue-resident memory T cells (Abou-Daya et al., 2021; Monti et al., 2008; Stegall et al., 1996).

### SUMMARY OF THE INVENTION

In some embodiments the invention relates to a cell engineered (i.e., modified) to induce a tolerogenic local microenvironment. In some embodiments the cells is an induced pluripotent cell; in some embodiments the cell is a stem cell. In some embodiments the cell is a progenitor or precursor cell, e.g., a beta cell progenitor cell or beta cell precursor cell. The cell is engineered to express one or more tolerogenic genes, e.g., one or more transgenes, and/or one or more immunomodulatory agents from at least one locus in the cell which is not substantially silenced during differentiation of the cell to a desired cell type. As used herein, a locus which is not substantially silenced is one which is measurably expressed after differentiation of the cell to the desired cell type. In some embodiments the locus is measurably expressed both during and after differentiation of the cell to the desired cell type. The invention relates both to the progenitor or precursor cell engineered as described and to the differentiated cell derived from the engineered precursor or progenitor cell and comprising the engineered locus. The invention further relates to methods of making the cell and methods of using the cell, e.g., in a method of cell therapy to treat a subject in need thereof.

In some embodiments the invention relates to a method of producing a hypoimmunogenic cell comprising engineering a stem cell or progenitor cell to express one or more tolerogenic transgenes from a locus in the stem cell or progenitor cell which is not silenced during differentiation of the cell to a desired cell type and exposing the stem cell or progenitor cell to conditions suitable for the stem cell or progenitor cell to differentiate to a desired cell type. The invention also relates to cells produced by this method, including the engineered stem cell, the engineered progenitor cell, and the engineered differentiated cell. In some embodiments the stem cell is an induced pluripotent cell. In some embodiments the progenitor cell is a beta cell progenitor cell. In some embodiments the desired cell type is a beta cell.

In some aspects the locus is the locus of a housekeeping gene. In some embodiments the locus is constitutively expressed in all cells of the islet, e.g., the human islet. In some embodiments the locus is constitutively expressed in beta cells, e.g., human beta cells. In some embodiments the housekeeping gene is selected from the group consisting of actin, ubiquitin, and GAPDH.

In some aspects the one or more tolerogenic agents/transgenes are selected from the group consisting of PDL1, HLA-E/G, CD47, SERPINB9, CCL21, FASL, CD200, MFGE8, CD55, CD46, HLS-G single chain fusion, soluble PDL1-Ig and CTLA4-Ig. In some aspects the tolerogenic agent is HLA-E single chain fusion.

In some embodiments the single chain fusion is loaded with a peptide capable of activating NK cells, e.g., through interaction with inhibitory receptor NKG2A and/or NKG2C. In some embodiments the peptide is selected from the group consisting of: VMAPRTLLL (SEQ ID NO: 2), VMAPRTLL (SEQ ID NO: 3), VMAPRTLFL (SEQ ID NO: 4), VMAPRTLVL (SEQ ID NO: 5), VMAPRTLIL (SEQ ID NO: 6), IMAPRTLVL (SEQ ID NO: 7), VMPPRTLLL (SEQ ID NO: 8), VMAPRTVLL (SEQ ID NO: 9), VTAPRTLLL (SEQ ID NO: 10), VTAPRTVLL (SEQ ID NO: 11), VMAPRTLTL (SEQ ID NO: 12) and VMAPRALLL (SEQ ID NO: 13).

In other aspects the invention relates to a method of mediating localized immune tolerance comprising engineering a stem cell or progenitor cell to express one or more immunomodulatory agents from a locus in the stem cell or progenitor cell which is not silenced during differentiation of the cell to a desired cell type and exposing the stem cell or progenitor cell to conditions suitable for the stem cell or progenitor cell to differentiate to a desired cell type, thereby producing a desired cell type that secretes said one or more immunomodulatory agents. The invention also relates to cells produced by this method, including the engineered stem cell, the engineered progenitor cell and the engineered differentiated cell. In some embodiments the stem cell is an induced pluripotent cell. In some embodiments the progenitor cell is a beta cell progenitor cell. In some embodiments the desired cell type is a beta cell. The invention also relates to cells produced by this method, including the engineered stem cell, the engineered progenitor cell, and the engineered differentiated cell.

In some aspects the locus is the locus of a housekeeping gene. In some embodiments the locus is constitutively expressed in all cells of the islet, e.g., the human islet. In some embodiments the locus is constitutively expressed in beta cells, e.g., human beta cells. In some embodiments the housekeeping gene is selected from the group consisting of actin, ubiquitin, and GAPDH. In some aspects, the one or more immunomodulatory agents is a cytokine. In some aspects, the one or more immunomodulatory agents is selected from the group consisting of IL-10, TGF-β and IL-2 and a modified IL-2.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1G: Generation of immune-evasive SC-islet cells. FIG. 1A is a schematic representation of the genetic-engineering strategy to generate hypoimmunogenic hESCs. FIG. 1B shows a FACS analysis of Nk×6.1/C-peptide SC-β cells derived from hypoimmunogenic hESCs (S6d14). FIG. 1C shows PDL1 and HLA-ABC expression on SC-β cells. Data is presented as MFI (gated on C-pep+/Nk×6.1+SC-β cells) normalized to mode. FIG. 1D shows HLA-ABC and HLA-E expression on SC-β cells. Data is presented as MFI (gated on C-pep+/Nk×6.1+SC-β cells) normalized to mode. FIG. 1E shows immunofluorescence staining of HLA-E, Nk×6.1 and C-peptide in CD49a+-enriched SC-β cells (S6d14). Size bars=100 μm. FIG. 1F is a schematic of in vivo SC-islet cell xeno-rejection assay. FIG. 1G shows a quantitative analysis of xenograft rejection. Data is presented as mean±SEM (n=5/group). Dashed line represents background luminescence.

FIGS. 2A-2I: In vitro co-culture of hypoimmunogenic SC-islet cells with allogeneic human immune cells. FIG. 2A shows a schematic of the in vitro immune SC-islet cell co-culture assay. FIG. 2B shows quantification of SC-islet cell survival when co-cultured with primary human PBMCs at a 1:1 ratio. Cell survival is presented as mean±SD (n=5). FIG. 2C shows quantification of SC-islet cell survival when co-cultured with primary human PBMCs at a 3:1 ratio. Cell survival is presented as mean±SD (n=5). FIG. 2D is a heatmap of T cell ligand gene expression in IFN-γ treated CD49a+SC-β cells. Ligand expression is presented as fold-change (log 2). FIG. 2E shows differential expression of T cell co-inhibitory ligands in IFN-γ treated CD49a+SC-β cells. Ligand expression is presented as fold-change (log 2). FIG. 2F shows differential expression of T cell co-activating ligands in IFN-γ treated CD49a+SC-β cells. Ligand expression is presented as fold-change (log 2). FIG. 2G shows quantification of SC-β cell survival when co-cultured with primary human CD56+NK cells. Cell survival is presented as mean±SD (n=5). FIG. 2H is a schematic of an in vivo NK cell cytotoxicity assay. FIG. 2I shows quantitative analysis of the in vivo NK cell assay. Data is presented as mean±SEM. Dashed line represents background luminescence.

FIGS. 3A-3G: Characterization of SC-islet cell-specific NK cell ligand profile. FIG. 3A shows a schematic of SC-β and SC-endothelial cell bulk RNA sequencing workflow. FIG. 3B is a heatmap of NK cell ligand expression in SC-β and SC-endothelial cells (−IFN-γ vs+IFN-γ). Ligand expression is presented as fold-change (log₂). FIG. 3C illustrates NK cell activating ligand expression on SC-β and SC-endothelial cells. Data is presented as MFI normalized to mode and is representative of three independent experiments. FIG. 3D shows NK cell inhibitory ligand expression on SC-β and SC-endothelial cells. Data is presented as MFI normalized to mode and is representative of three independent experiments. FIG. 3E is a schematic of SC-islet cell transplantation in diabetic humanized NSG-DKO mice.

FIG. 3F shows non-fasting blood glucose concentrations after SC-islet transplantation and PBMC injection. Data is presented as mean±SD (n=12/group). FIG. 3G illustrates in vivo GSIS pre- and post-PBMC injection. Plasma insulin concentration was measured t=0 and 30 min after glucose injection. Data is represented as mean±SD (n=5/group).

FIGS. 4A-4H: Immune-tolerizing SC-islet cells survive xeno-rejection. FIG. 4A is a schematic representation of the genetic-engineering strategy to generate immune-modulatory hESCs and of the GAP-2B10 HDR plasmid. FIG. 4B is a FACS analysis of Nk×6.1/C-peptide SC-β cells derived from immune-modulatory hESCs (S6d8). FIG. 4C shows in vitro GSIS of 2B10 SC-islet cells. Data is represented as mean±SD (n=4). FIG. 4D illustrates quantification of cytokine secretion from 2B10 SC-islet cells. Data is represented as mean±SD (n=2). FIG. 4E shows quantification of SC-islet cell survival when co-cultured with primary human PBMCs at a 1:1 ratio. Cell survival is presented as mean±SD (n=5). FIG. 4F shows in vivo bioluminescence imaging of B6/albino mice transplanted with 2B10 SC-islet cells (n=3/group). FIG. 4G shows quantitative analysis of in vivo graft survival in B6/albino mice. Data is presented as mean±SD (n=3/group). FIG. 4H shows immunostaining of 2B10 and WT SC-islet grafts showing presence of INS⁺ cells and T_{reg} recruitment at 5 weeks post transplantation. Size bars=100 μm.

FIGS. 5A-5H: FIG. 5A shows t-SNE projections of primary human islet cells. FIG. 5B shows t-SNE projections of GAPDH expression across the assigned populations. Cells are colored according to their assigned cluster. (Adapted from Segerstolpe et al., 2016). FIG. 5C is a schematic of homology directed repair plasmids for integration of Luc2 and PD-L1 at the GAPDH locus. FIG. 5D is a schematic of GAPDH-targeting Luc2 and peptide::B2M::HLA-E HDR plasmid. FIG. 5E is a schematic of the peptide::B2M::HLA-E long-chain fusion. FIG. 5F shows a PD1-Fc binding assay. PD-L1 and WT SC-islet cells were dissociated and stained with a 2-fold dilution series of PE-conjugated human and mouse PD1-Fc. Data is presented as MFI normalized to mode. FIG. 5G shows a FACS sorting strategy of HLA-ABC−/− hESCs. FIG. 5H shows a FACS gating strategy for quantification of Nk×6.1+/C-pep+SC-β cells following in vitro differentiation.

FIGS. 6A-6H: FIG. 6A shows FACS profiling of HLA-A2 status of 5 PBMC donors. FIG. 6B shows a FACS gating strategy of CD4⁺ and CD8⁺ T cells and CD56⁺ NK cells in PBMCs enriched from human apheresis leukoreductions. Plots are representative of 5 donors. FIG. 6C shows quantification of SC-islet cell survival when co-cultured with purified human CD8+ T cells at a 1:1 ratio. Cell survival is presented as mean±SD (n=5). FIG. 6D shows quantification of SC-islet cell survival when co-cultured with purified human CD8+ T cells at a 3:1 ratio. Cell survival is presented as mean±SD (n=5). FIG. 6E shows quantification of SC-islet cell survival when co-cultured with purified human CD4⁺ T cells at a 1:1 ratio. Cell survival is presented as mean±SD (n=5). FIG. 6F shows quantification of SC-islet cell survival when co-cultured with purified human CD4+ T cells at a 3:1 ratio. Cell survival is presented as mean±SD (n=5). FIG. 6G shows a FACS gating strategy for CD4+ T cells enriched from human apheresis leukoreductions. Plots are representative of 5 donors. FIG. 6H shows a FACS gating strategy for CD8⁺ T cells enriched from human apheresis leukoreductions. Plots are representative of 5 donors.

FIGS. 7A-7G: FIG. 7A shows quantification of SC-β cell survival when co-cultured with NK92mi cells. K562 and Raji cells were used as positive and negative controls, respectively. Cell survival is presented as mean±SD (n=5). FIG. 7B shows NKG2A/NKG2C expression on NK92mi cells. FIG. 7C shows a FACS gating strategy for CD56+NK cells enriched from human apheresis leukoreductions. Gates for specific population include all CD56+NK cells (red), CD56^{high} (green) and CD56^{dim} (pink). Plots are representative of 5 donors. FIG. 7D shows quantitative analysis of CD56 expression on enriched primary human NK cells. Data is presented as % CD56 expression (n=5). FIG. 7E shows an in vivo NK cell assay. Bioluminescence imaging was performed on day 1 and 5 post-transplantation. FIG. 7F is a schematic of SC-endothelial cell differentiation protocol. FIG. 7G shows FACS analysis of the endothelial cell marker CD31 in SC-endothelial cells derived from WT and B2M−/− hESCs.

### DETAILED DESCRIPTION OF THE INVENTION

Immunological protection of transplanted stem cell-derived islet (SC-islet) cells is yet to be achieved without chronic immunosuppression or encapsulation. Existing genetic engineering approaches to produce hypoimmunogenic SC-islet cells have so far shown variable results. It is shown herein that targeting the human leukocyte antigens (HLAs) and PD-L1 alone do not sufficiently protect SC-islet cells from xeno- or allo-rejection. Described herein are genetically engineered SC-islet cells that secrete the cytokines IL-10, TGF-β and modified IL-2 such that they promote a tolerogenic local microenvironment by activating and expanding regulatory T cells (Tregs). These cytokine-secreting human SC-islet cells prevented xeno-rejection for up to 9 weeks post-transplantation in B6/albino mice. Thus, hESCs engineered to induce a tolerogenic local microenvironment may represent a source of replacement SC-islet cells that do not require encapsulation or immunosuppression for diabetes cell replacement therapy. The described cell modifications can be used alone or in addition to other approaches to produce hypoimmunogenic SC-islet cells.

To address the issue of transgene silencing during differentiation into SC-islet cells, a transgene targeting strategy was employed herein that makes use of the constitutively expressed GAPDH gene in primary human islets and SC-islet cells (Gerace et al., 2021; Sintov et al., 2021). We generated hypoimmunogenic SC-islet cells that over-express PD-L1 and the HLA-E long-chain fusion in both HLA-competent and deficient settings. In these studies, PD-L1 over-expression did not protect SC-islet cells from xeno-rejection, and over-expression of the HLA-E single-chain fusion was not required to inhibit primary human NK cells. In addition to immune evasion, we also address immune modulation and show that constitutive secretion of IL-10, TGF-β and the IL-2 mutein does not impair SC-islet cell function in vitro and does provide protection of SC-islet cells from xeno-rejection for up to 9 weeks after transplantation. Thus, these immune-modulating SC-islet cells represent another step forward in providing a source of islets for the treatment of T1D with the long-term aim of eliminating the need for encapsulation or systemic immunosuppression.

The utility of an immune-evasive/tolerogenic islet cell replacement therapy relies on the ability to maintain transgene expression throughout cell differentiation and after transplantation. Thus, to analyze the effect of various gene-editing strategies for the immune protection of SC-islet cells, SC-islet cells were engineered to constitutively express tolerogenic molecules including PD-L1 (Castro-Gutierrez et al., 2021; Yoshihara et al., 2020) and the HLA-E long-chain fusion (Gornalusse et al., 2017) from the GAPDH locus, both in a HLA-competent and HLA-deficient background. We report the lack of a xeno-protective effect of PD-L1 over-expression in SC-islet cells. This may be explained by species-specific differences in PD-L1/PD-1 binding (Viricel et al., 2015), and the fact that human and mouse PD-1 share only 60% homology at the amino acid level (Finger et al., 1997). In fact, a decrease in binding of soluble mouse PD-1 to human PD-L1 expressed on SC-islet cells was observed. While these results suggest that over-expression of human PD-L1 is not sufficient to overcome xeno-rejection, PD-L1 over-expression may still be useful in an allogeneic setting.

In concordance with previous studies, HLA-deficient SC-islet cells were resistant to PBMC cytotoxicity in vitro (Han et al., 2019; Leite et al., 2022). We also found that SC-β cells modulate their T cell ligand profile in response to partial inflammatory stimulus. While we and others have attempted to exploit the PD-L1/PD-1 T cell signaling axis to engineer immune-evasive SC-islet cells (Yoshihara et al., 2020), our transcript analysis of T cell ligands in SC-β cells suggests that exploiting the LGALS9/TIM-3 signaling axis may also be of interest. In fact, like PD-L1, LGALS9 is frequently upregulated in cancer cells where it contributes to tumor progression by inhibition of T cell function (Heusschen et al., 2013; Yang et al., 2021). Additionally, we identified the T cell activating ligands HVEM, CD40 and BTN3A1 as potential targets to knock-out in SC-islet cells to further influence T cell function.

Work described herein also showed that HLA-deficient SC-islet cells are resistant to pre-activated NK cell cytotoxicity both in vitro and in vivo, and that over-expression of the HLA-E long-chain fusion does not provide any additional protective benefit. This is due to the SC-islet cell-specific lack of NK cell activating ligands such as the MIC and ULBP proteins, which are expressed in SC-endothelial cells, and may explain why SC-endothelial cells are susceptible to pre-activated NK cell cytotoxicity while SC-islet cells are resistant (Deuse et al., 2021). However, since many immunodeficient mouse models lack critical components for NK cell survival and function such as SIRPα and IL-15 (Herndler-Brandstetter et al., 2017), they do not support long-term engraftment of human NK cells, and we cannot exclude the possibility of HLA-deficient SC-islet cell destruction in a mouse model that better supports NK cell engraftment. Furthermore, while transplantation of HLA-deficient SC-islet cells was able to normalize blood glucose levels in diabetic mice, after PBMC injection the cells were eventually rejected, albeit with delayed rejection kinetics. This could be explained by the presence of other immune cell subsets such as macrophage, monocytes and dendritic cells that may play a role in indirect allograft rejection (Oberbarnscheidt et al., 2014; Wyburn et al., 2005; Zhuang et al., 2016). Ultimately, this highlights the limitations of exclusively using in vitro immune cell co-culture assays to assess the effect of genetic modification on the protection SC-islet cells from immune destruction (Castro-Gutierrez et al., 2021; Leite et al., 2022).

Finally, work described here shows for the first time that SC-islet cells engineered to secrete modified IL-2, TGF-β and IL-10 are protected against xeno-rejection. This finding has several important implications. First, since 3 cells are professional secretory cells with a significant translatory demand, it demonstrates that SC-islet cells can be co-opted to secrete other proteins while maintaining their designed function (Lim et al., 2020). Second, the intrinsic ligand profile of the desired cell type should be considered when determining the set of genetic modifications required to generate immune-evasive cells, as some cell types may not require extensive genetic manipulation. Since the safety of immune-evasive/immune-tolerizing cell therapies is critical to their clinical translation, ideally the genetically engineered product should be generated with the least number of genetic perturbations to limit off-target events and chromosomal instability. Third, these results provide validation for the use of immune-tolerizing approaches (either alone or in conjunction with immune-evasion) as a method to protect SC-islet cells from the immune system. A variation of this approach may be to include some, but not all, cells in the transplant that secrete these tolerizing molecules. Overall, this approach may eliminate the need for encapsulation or immunosuppression, a long-standing goal of the islet transplantation field.

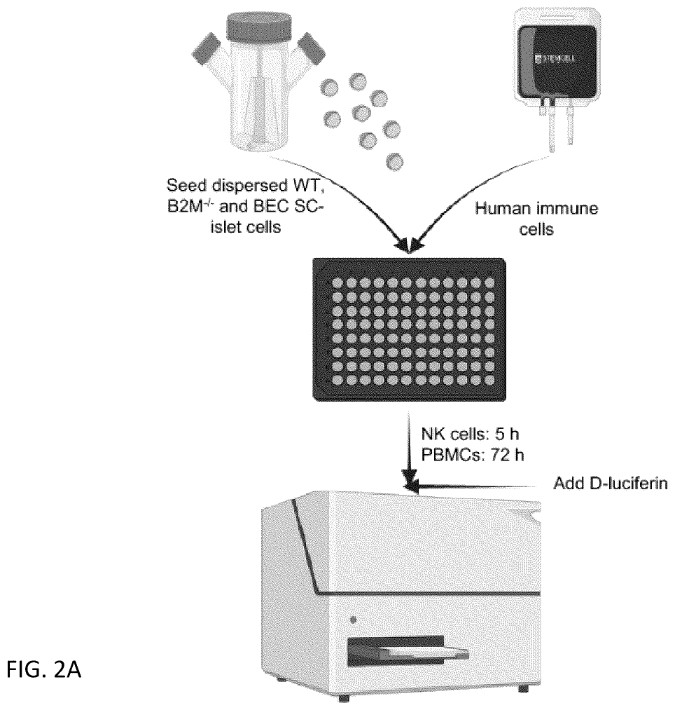

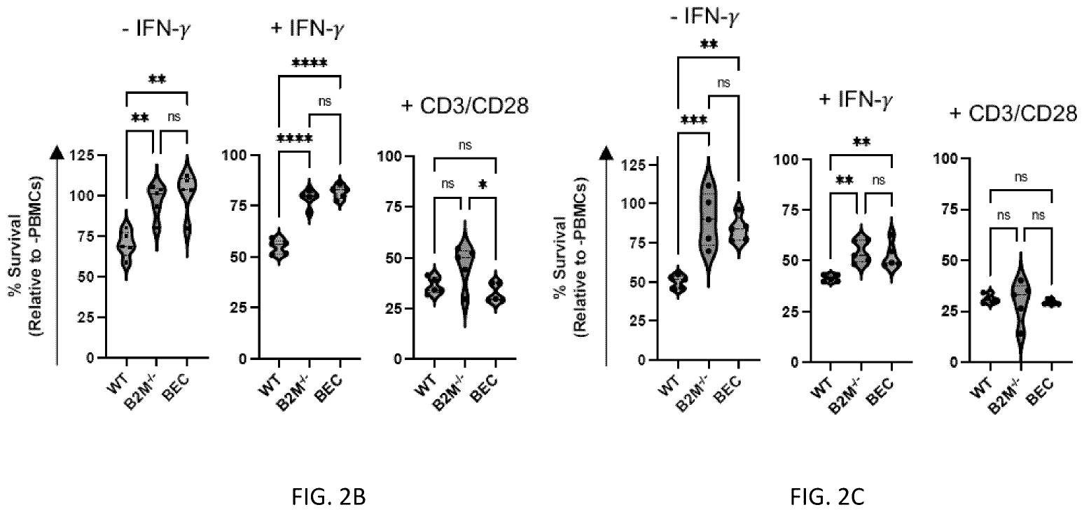

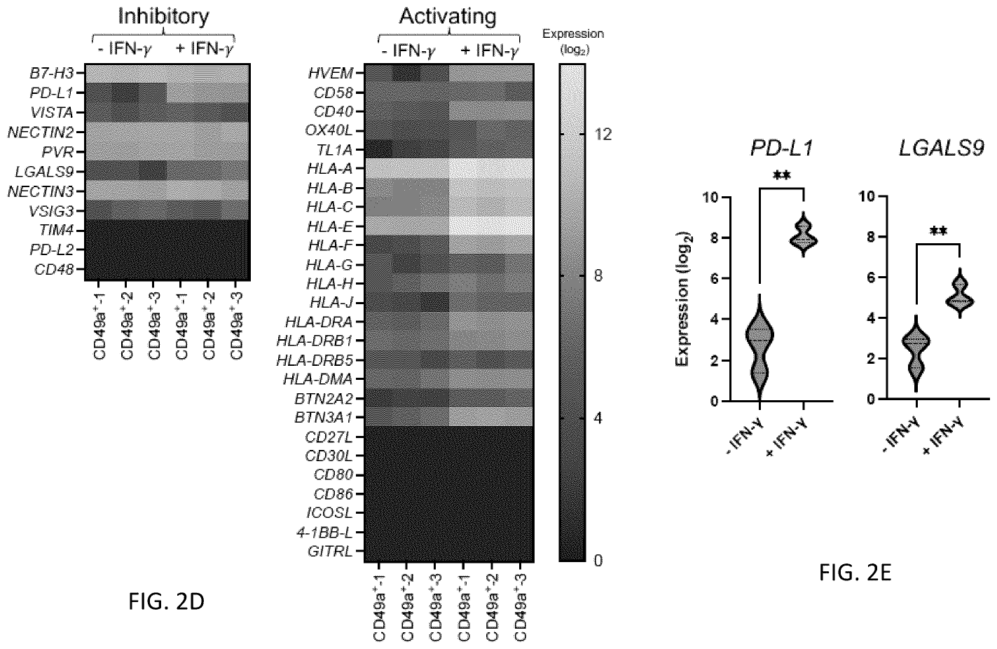

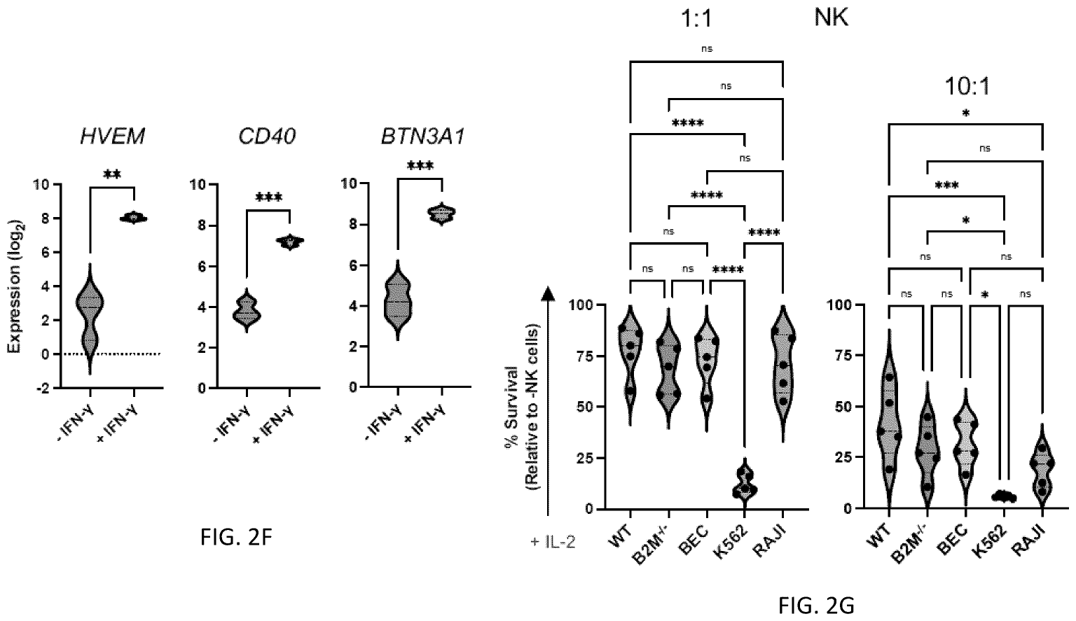

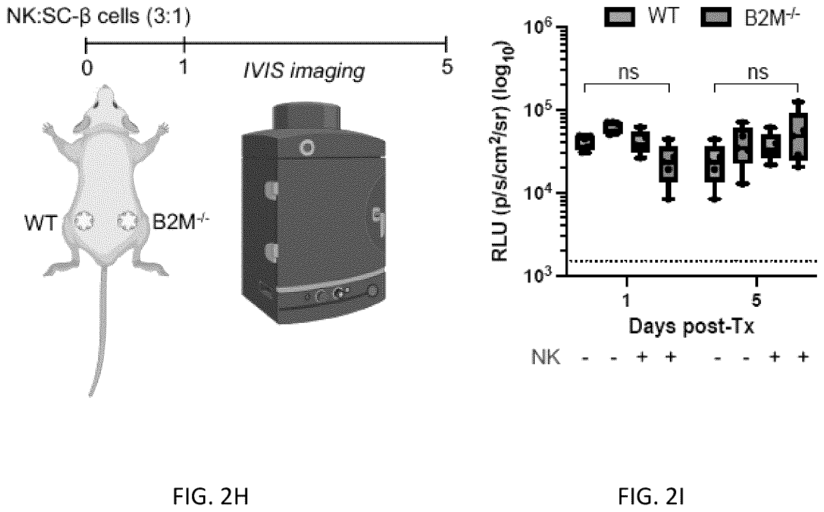

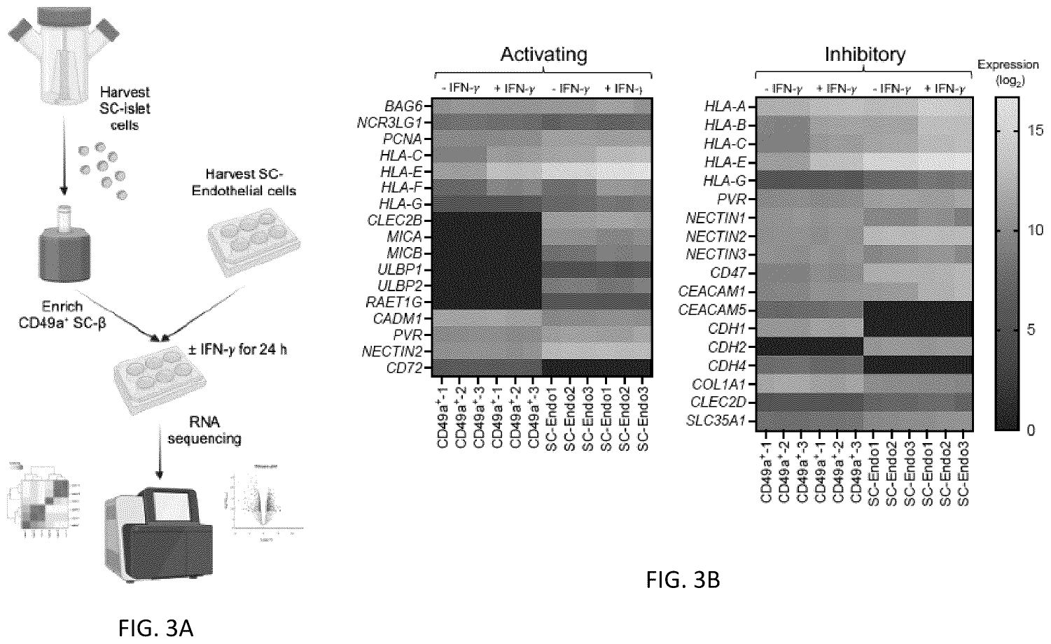

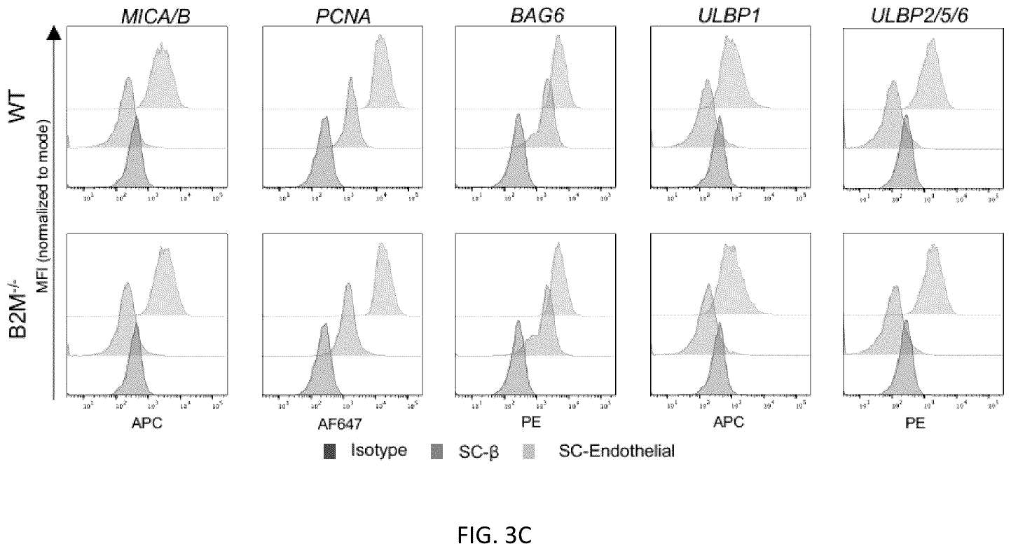

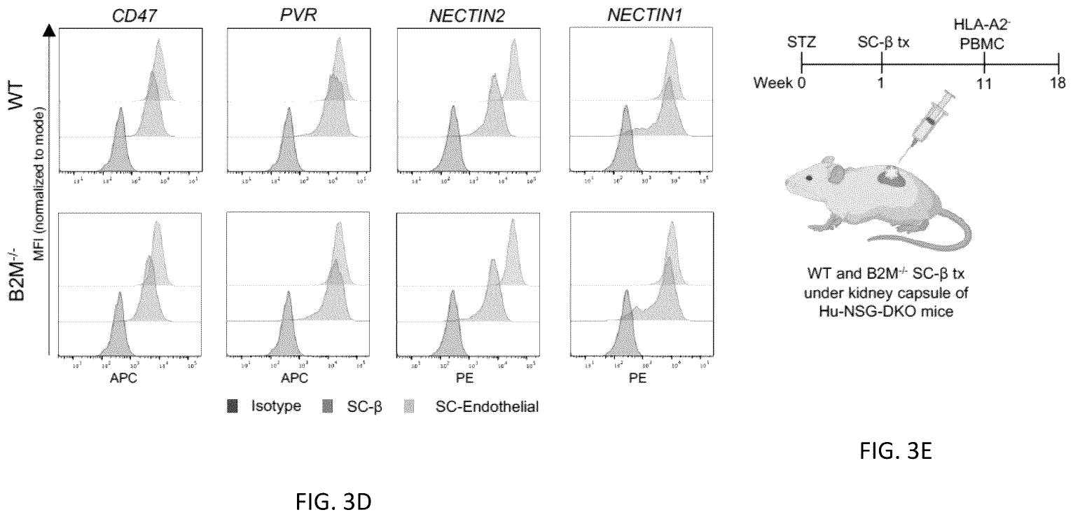

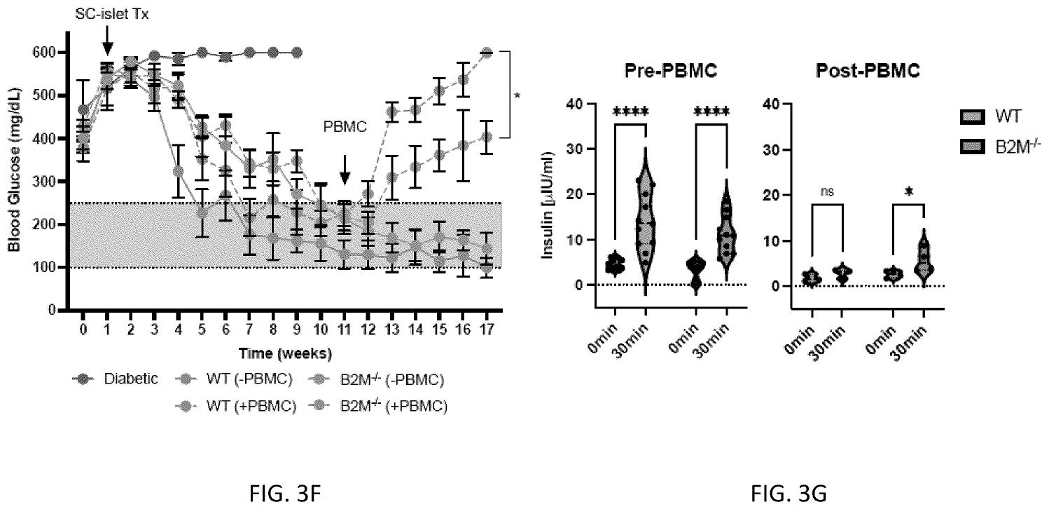

Although HLA-deficient SC-islet cells possessed improved survival in in vitro PBMC co-culture assays (FIGS. 2B and 2C), these results did not translate in vivo. Choosing an appropriate humanized mouse model is essential to evaluate immune-evasion/immune-tolerizing genetic engineering strategies. Many PBMC humanized mouse models such as that used in this study do not fully recapitulate human allograft rejection since they bias CD3+ cell engraftment and lack other immune cell subsets. Thus, while HLA-deficient SC-islets demonstrated delayed rejection in the PBMC humanized mouse model (FIG. 3F), the possibility that in CD34+ or BLT mice (humanized mouse models that better recapitulate human immune components) these cells would be rejected earlier cannot be excluded. For these reasons, validating the protective effect of immune-evasion/tolerizing genetic-engineering strategies should ideally be conducted in humanized mouse models that more accurately reflect allograft rejection. While xeno-rejection does not mimic allo-rejection, a xeno-rejection model was used to evaluate this immune-tolerizing genetic-engineering strategy, as it is a stronger model of immune rejection and eliminates the immune cell bias of humanized mouse models.

By using such a strong model of graft rejection, additional immune barriers are introduced that require complex genetic-engineering approaches as a solution. This means combining gene knock-outs and knock-ins to generate the desired immune-evasive/tolerizing cell product. Knock-in of multiple genes at a specific locus in the genome requires large homology directed repair templates, which is associated with poor integration efficiency and recovery of genetically unstable clones. Exploring the possibility of introducing tolerogenic molecules at multiple constitutively expressed loci could reduce the size of HDR templates and result in the recovery of genetically stable homozygous knock-in clones. Striking a balance between HDR length and the number of editing events may permit the introduction of more foreign genetic material into the genome without destabilizing effects.

Furthermore, since immune-tolerizing SC-islets secrete cytokines from all cells within the islet, there is a risk that the concentration of constitutively secreted cytokines may result in chronic immunosuppression. Thus, it is important to evaluate the immune status of mice receiving immune-tolerizing SC-islet cells as chronic immunosuppression should be avoided. However, if transplantation of immune-tolerizing SC-islets does result in chronic immunosuppression, the ability to enrich for specific endocrine cell populations may allow us to adjust the dose of cytokine-secretion by generating designer islets composed of cytokine-secreting and non-cytokine secreting endocrine cells such that localized graft tolerance is achieved.

### Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell leads to the pancreatic pathway, where ^{˜}98% of the cells become exocrine, ductular, or matrix cells, and ^{˜}2% become endocrine cells.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods described herein can be performed both in vivo and in vitro.

As used herein, the term “adult cell” refers to a cell found throughout the body after embryonic development.

The term “endoderm cell” as used herein refers to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of respiratory and digestive tracts (e.g. the intestine), the liver and the pancreas.

The term “a cell of endoderm origin” as used herein refers to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) develop from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates.

The terms “stem cell-derived β cell”, “SC-β cell”, and “mature SC-β cell” refer to cells (e.g., pancreatic β cells) that display at least one marker indicative of a pancreatic β cell, express insulin, and display a GSIS response characteristic of an endogenous mature β cell. In some embodiments, the “SC-β cell” comprises a mature pancreatic β cell. It is to be understood that the SC-β cells need not be derived (e.g., directly) from stem cells. Moreover, it should be understood that an SC-β cell of the invention is a non-native, i.e., non-naturally occurring, non-endogenous, cell and has at least one characteristic that is different from a native/naturally-occurring/endogenous cell. Examples of SC-β cells, and methods of obtaining such SC-β cells, are described in WO 2015/002724, WO 2014/201167, Millman et al., 2016, and Pagliuca et al., 2014, all of which are incorporated herein by reference in their entirety. In some embodiments, the “SC-β cells” are hypoimmunogenic stem cell-derived beta cells, e.g., beta cells that generate limited or no immune reaction.

The term “exocrine cell” as used herein refers to a cell of an exocrine gland, i.e. a gland that discharges its secretion via a duct. In particular embodiments, an exocrine cell refers to a pancreatic exocrine cell, which is a pancreatic cell that produces enzymes that are secreted into the small intestine. These enzymes help digest food as it passes through the gastrointestinal tract. Pancreatic exocrine cells are also known as islets of Langerhans, that secrete two hormones, insulin and glucagon.

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refers to a pluripotent stem cell artificially derived (e.g., induced or by complete reversal) from a non-pluripotent cell, typically an adult somatic cell, for example, by inducing a forced expression of one or more genes.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a stem cell derived cell described herein.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic acid into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification), the modification is not suitable for the methods and compositions described herein.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, and are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “lineages” as used herein describes a cell with a common ancestry or cells with a common developmental fate. For example, in the context of a cell that is of endoderm origin or is “endodermal linage” this means the cell was derived from an endoderm cell and can differentiate along the endoderm lineage restricted pathways, such as one or more developmental lineage pathways which give rise to definitive endoderm cells, which in turn can differentiate into liver cells, thymus, pancreas, lung and intestine.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term a “variant” in referring to a polypeptide could be, e.g., a polypeptide at least 80%, 85%, 90%, 95%, 98%, or 99% identical to full length polypeptide. The variant could be a fragment of full length polypeptide. The variant could be a naturally occurring splice variant. The variant could be a polypeptide at least 80%, 85%, 90%, 95%, 98%, or 99% identical to a fragment of the polypeptide, wherein the fragment is at least 50%, 60%, 70%, 80%, 85%, 90%, 95%, 98%, or 99% as long as the full length wild type polypeptide or a domain thereof having an activity of interest. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature (full length) polypeptide, by which is meant a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, by which is meant that it contains portions from two or more different species. In some embodiments wherein a protein is produced other than by purifying it from cells that naturally express it, the protein is a derivative, by which is meant that the protein comprises additional sequences not related to the protein so long as those sequences do not substantially reduce the biological activity of the protein.

The term “functional fragments” as used herein is a polypeptide having an amino acid sequence which is smaller in size than, but substantially homologous to the polypeptide it is a fragment of, and where the functional fragment polypeptide sequence is about at least 50%, or 60% or 70% or 80% or 90% or 100% or greater than 100%, for example 1.5-fold, 2-fold, 3-fold, 4-fold or greater than 4-fold effective biological action as the polypeptide from which it is a fragment of. Functional fragment polypeptides may have additional functions that can include decreased antigenicity, increased DNA binding (as in transcription factors), or altered RNA binding (as in regulating RNA stability or degradation).

The term “vector” refers to a carrier DNA molecule into which a DNA sequence can be inserted for introduction into a host cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g., promoters, enhancers, and termination elements) in an expression vector. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g., EPV and EBV vectors.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances, the promoter sequence is recognized by the synthetic machinery of the cell, or introduced synthetic machinery, required for initiating transcription of a specific gene.

As used herein, the term “transcription factor” refers to a protein that binds to specific parts of DNA using DNA binding domains and is part of the system that controls the transfer (or transcription) of genetic information from DNA to RNA. As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection,” and the marker is said to be “useful for positive selection.” Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

A “reporter gene” as used herein encompasses any gene that is genetically introduced into a cell that adds to the phenotype of the stem cell. Reporter genes as disclosed in this invention are intended to encompass fluorescent, luminescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny. A reporter gene is generally operatively linked to sequences that regulate its expression in a manner dependent upon one or more conditions which are monitored by measuring expression of the reporter gene. In some cases, expression of the reporter gene may be determined in live cells. Where live cell reporter gene assays are used, reporter gene expression may be monitored at multiple time points, e.g., 2, 3, 4, 5, 6, 8, or 10 or more time points. In some cases, where a live cell reporter assay is used, reporter gene expression is monitored with a frequency of at least about 10 minutes to about 24 hours, e.g., 20 minutes, 1 hour, 2 hours, 3 hours, 4 hours, 5 hours, 6 hours, 7 hours, 8 hours, 9 hours, 10 hours, 12 hours, 18 hours, or another frequency from any integer between about 10 minutes to about 24 hours.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g., cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill or injured, or at increased risk of becoming ill relative to an average member of the population and in need of such attention, care, or management.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

As used herein, the terms “administering,” “introducing” and “transplanting” are used interchangeably in the context of the placement of cells of the invention into a subject, by a method or route which results in at least partial localization of the introduced cells at a desired site. The cells can be implanted directly to the pancreas or gastrointestinal tract, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years. In some instances, the cells can also be administered subcutaneously, for example, in a capsule (e.g., microcapsule) to maintain the implanted cells at the implant location and avoid migration of the implanted cells.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and intrasternal injection and infusion. The phrases “systemic administration,” “administered systemically”, “peripheral administration” and “administered peripherally” as used herein mean the administration of stem cell-derived cells and/or their progeny and/or compound and/or other material other than directly into the central nervous system, such that it enters the animal's system and, thus, is subject to metabolism and other like processes, for example, subcutaneous administration.

The term “tissue” refers to a group or layer of specialized cells which together perform certain special functions. The term “tissue-specific” refers to a source of cells from a specific tissue.

The terms “decrease,” “reduced,” “reduction,” “decrease,” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

### Stem Cells

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

While certain embodiments are described below in reference to the use of stem cells, germ cells may be used in place of, or with, the stem cells to provide at least one differentiated cell, using similar protocols as the illustrative protocols described herein. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

ES cells, e.g., human embryonic stem cells (hESCs) or mouse embryonic stem cells (mESCs), with a virtually endless replication capacity and the potential to differentiate into most cell types, present, in principle, an unlimited starting material to generate the differentiated cells for clinical therapy (stemcells.nih.gov/info/scireport/2006report.htm, 2006).

hESC cells, are described, for example, by Cowan et al. (N Engl. J. Med. 350:1353, 2004) and Thomson et al. (Science 282:1145, 1998); embryonic stem cells from other primates, Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998) may also be used in the methods disclosed herein. mESCs, are described, for example, by Tremml et al. (Curr Protoc Stem Cell Biol. Chapter 1:Unit 1C.4, 2008). The stem cells may be, for example, unipotent, totipotent, multipotent, or pluripotent. In some examples, any cells of primate origin that are capable of producing progeny that are derivatives of at least one germinal layer, or all three germinal layers, may be used in the methods disclosed herein.

In certain examples, ES cells may be isolated, for example, as described in Cowan et al. (N Engl. J. Med. 350:1353, 2004) and U.S. Pat. No. 5,843,780 and Thomson et al., Proc. Natl. Acad. Sci. USA 92:7844, 1995. For example, hESCs cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hESCs include their pluripotent derivatives, such as primitive ectoderm-like (EPL) cells, as outlined, for example, in WO 01/51610 (Bresagen). hESCs can also be obtained from human pre-implantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses can be isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers. After 9 to 15 days, inner cell mass-derived outgrowths can be dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology can be individually selected by micropipette, mechanically dissociated into clumps, and replated. ES-like morphology is characterized as compact colonies with apparently high nucleus to cytoplasm ratio and prominent nucleoli. Resulting hESCs can then be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some examples, clump sizes of about 50 to 100 cells are optimal. mESCs cells can be prepared from using the techniques described by e.g., Conner et al. (Curr. Prot. in Mol. Biol. Unit 23.4, 2003).

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, as outlined in WO 01/51610 (Bresagen).

Alternatively, in some embodiments, hES cells can be obtained from human preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (^{˜}200 U/mL; Gibco) or by selection of individual colonies by micropipette. Clump sizes of about 50 to 100 cells are optimal.

In some embodiments, human Embryonic Germ (hEG) cells are pluripotent stem cells which can be used in the methods as disclosed herein to differentiate into primitive endoderm cells. hEG cells can be prepared from primordial germ cells present in human fetal material taken about 8-11 weeks after the last menstrual period. Suitable preparation methods are described in Shamblott et al., Proc. Natl. Acad. Sci. USA 95:13726, 1998 and U.S. Pat. No. 6,090,622, which is incorporated herein in its entirety by reference.

Briefly, genital ridges are processed to form disaggregated cells. EG growth medium is DMEM, 4500 mg/L D-glucose, 2200 mg/L mM NaHCO₃; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/mL human recombinant bFGF (Genzyme); and 10 μM forskolin (in 10% DMSO). Ninety-six well tissue culture plates are prepared with a sub-confluent layer of feeder cells (e.g., STO cells, ATCC No. CRL 1503) cultured for 3 days in modified EG growth medium free of LIF, bFGF or forskolin, inactivated with 5000 rad γ-irradiation ^{˜}0.2 mL of primary germ cell (PGC) suspension is added to each of the wells. The first passage is done after 7-10 days in EG growth medium, transferring each well to one well of a 24-well culture dish previously prepared with irradiated STO mouse fibroblasts. The cells are cultured with daily replacement of medium until cell morphology consistent with EG cells is observed, typically after 7-30 days or 1-4 passages.

In certain examples, the stem cells can be undifferentiated (e.g., a cell not committed to a specific lineage) prior to exposure to at least one maturation factor according to the methods as disclosed herein, whereas in other examples it may be desirable to differentiate the stem cells to one or more intermediate cell types prior to exposure of the at least one maturation factor (s) described herein. For example, the stem cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g., derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hESI (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into stem cell-derived cells did not involve destroying a human embryo.

In another embodiment, the stem cells can be isolated from tissue including solid tissue. In some embodiments, the tissue is skin, fat tissue (e.g., adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g., human, equine, bovine, porcine, canine, feline, rodent, e.g., mice, rats, hamster, primate, etc. In some embodiments, a human embryo was not destroyed for the source of pluripotent cell used on the methods and compositions as disclosed herein.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subjects adipose tissue, for example using the CELUTION™ SYSTEM from Cytori, as disclosed in U.S. Pat. Nos. 7,390,484 and 7,429,488 which is incorporated herein in its entirety by reference.

In some embodiments, human umbilical cord blood cells (HUCBC) are useful in the methods as disclosed herein. Human UBC cells are recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e., acute lymphoid leukemia, acute myeloid leukemia, chronic myeloid leukemia, myelodysplastic syndrome, and neuroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985J. Immunol. 134:1493-1497). Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985J. Immunol. 134:1493-1497 Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

In another embodiment, pluripotent cells are cells in the hematopoietic micro-environment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

In another embodiment, pluripotent cells are present in embryoid bodies are formed by harvesting ES cells with brief protease digestion and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

### Cloning and Cell Culture

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, for example, in Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM β-mercaptoethanol. Just before use, human bFGF is added to 4 ng/mL (WO 99/20741, Geron Corp.). Traditionally, ES cells are cultured on a layer of feeder cells, typically fibroblasts derived from embryonic or fetal tissue.

Pluripotent SCs can be maintained in an undifferentiated state even without feeder cells. The environment for feeder-free cultures includes a suitable culture substrate, particularly an extracellular matrix such as Matrigel® or laminin. Typically, enzymatic digestion is halted before cells become completely dispersed (say, ^{˜}5 min with collagenase IV). Clumps of ^{˜}10 to 2,000 cells are then plated directly onto the substrate without further dispersal.

Feeder-free cultures are supported by a nutrient medium containing factors that support proliferation of the cells without differentiation. Such factors may be introduced into the medium by culturing the medium with cells secreting such factors, such as irradiated (^{˜}4,000 rad) primary mouse embryonic fibroblasts, telomerized mouse fibroblasts, or fibroblast-like cells derived from pPS cells. Medium can be conditioned by plating the feeders at a density of ^{˜}5-6×10⁴ cm⁻² in a serum free medium such as KO DMEM supplemented with 20% serum replacement and 4 ng/mL bFGF. Medium that has been conditioned for 1-2 days is supplemented with further bFGF and used to support pluripotent SC culture for 1-2 days. Features of the feeder-free culture method are further discussed in International Patent Publication WO 01/51616; and Xu et al., Nat. Biotechnol. 19:971, 2001.

Under the microscope, ES cells appear with high nuclear/cytoplasmic ratios, prominent nucleoli, and compact colony formation with poorly discernable cell junctions. Primate ES cells express stage-specific embryonic antigens (SSEA) 3 and 4, and markers detectable using antibodies designated Tra-1-60 and Tra-1-81 (Thomson et al., Science 282:1145, 1998). Mouse ES cells can be used as a positive control for SSEA-1, and as a negative control for SSEA-4, Tra-1-60, and Tra-1-81. SSEA-4 is consistently present human embryonal carcinoma (hEC) cells. Differentiation of pluripotent SCs in vitro results in the loss of SSEA-4, Tra-1-60, and Tra-1-81 expression, and increased expression of SSEA-1, which is also found on undifferentiated hEG cells.

### Methods of Generating Hypoimmunogenic Cells

Aspects of the disclosure relate to generating hypoimmunogenic cells. The hypoimmunogenic cells may include islet or beta cells, e.g., stem-cell derived beta cells. In some embodiments, a stem cell or progenitor cell is engineered to express one or more tolerogenic transgenes from a locus in the cell, where the tolerogenic transgene is not silenced during differentiation of the cell to a desired cell type. The engineered stem cell or progenitor cell may be exposed to conditions suitable for the cell to differentiate to a desired cell type, e.g., an SC-islet or SC-beta cell.

In some embodiments, the stem cell or progenitor cell is a pluripotent stem cell, e.g., an iPSC, a pancreatic progenitor, a non-native endocrine cell, or a beta cell progenitor cell. In some embodiments, the stem cell or progenitor cell may be engineered to express one or more tolerogenic transgenes from a locus in the stem cell or progenitor cell wherein the tolerogenic transgene will not be silenced during differentiation. Non-limiting examples of tolerogenic transgenes include PDL1, HLA-E/G, CD47, SERPINB9, CCL21, FASL, CD200, MFGE8, CD55, CD46, HLS-G single chain fusion, soluble PDL1-Ig and CTLA4-Ig. In one embodiment, the tolerogenic agent is HLA-E single chain fusion. The single chain fusion may be loaded with a peptide capable of activating NK cells, e.g., through interacting with the inhibitor receptor NKG2A and/or NKG2C. In some embodiments, the peptide may be VMAPRTLL (SEQ ID NO: 3), VMAPRTLFL (SEQ ID NO: 4), VMAPRTLVL (SEQ ID NO: 5), VMAPRTLIL (SEQ ID NO: 6), IMAPRTLVL (SEQ ID NO: 7), VMPPRTLLL (SEQ ID NO: 8), VMAPRTVLL (SEQ ID NO: 9), VTAPRTLLL (SEQ ID NO: 10), VTAPRTVLL (SEQ ID NO: 11), VMAPRTLTL (SEQ ID NO: 12) and/or VMAPRALLL (SEQ ID NO: 13).

In some embodiments, the stem cell or progenitor cell may be engineered to express one or more immunomodulatory agents from a locus in the stem cell or progenitor cell where the one or more immunomodulatory agents will not be silenced during differentiation of the cell. In some embodiments, the immunomodulatory agent comprises a cytokine. In certain embodiments, immunomodulatory agents comprises IL-10, TGF-β, and/or IL-2. In one embodiment, the immunomodulatory agent is a cytokine. In one embodiment, the immunomodulatory agent is IL-10. In one embodiment, the immunomodulatory agent is TGF-β. In one embodiment, the immunomodulatory agent is IL-2.

In some embodiments modifying the expression of one or more genes in stem or progenitor cells occurs using any gene editing tool known to those of skill in the art (e.g., TALENS, CRISPR, etc.). In some embodiments, the gene editing tool is delivered to the stem cells using a retrovirus (e.g., a lentivirus). In some embodiments, the one or more genes may be targeted using gene editing (e.g., CRISPR) to modulate expression of the one or more genes.

In some embodiments, the engineered stem cell or progenitor cell, e.g., an iPSC cell or a beta cell progenitor cell, is differentiated into a beta cell, e.g., a SC-beta cell. In some embodiments, the engineered stem cell or progenitor cell, e.g., an iPSC cell or a beta cell progenitor cell, is differentiated into an islet, e.g., a SC-islet. In some embodiments, the engineered stem cell or progenitor cell is differentiated into a desired cell type, e.g., a beta cell, that secretes one or more immunomodulatory agents from a locus in the cell.

In some embodiments, the stem cell or progenitor cell is engineered to express one or more tolerogenic transgenes from a locus in the stem cell or progenitor cell. In some embodiments, the stem cell or progenitor cell is engineered to express one or more immunomodulatory agents from a locus in the stem cell or progenitor cell. In some embodiments, the locus is constitutively expressed in all cells of an islet, e.g., a human islet. In some embodiments, the locus is constitutively expressed in beta cells, e.g., in human beta cells. The locus may be the locus of a housekeeping gene. In some embodiments, the housekeeping gene includes actin, ubiquitin, and/or GAPDH. In one embodiment, the housekeeping gene is GAPDH.

At least one hypoimmunogenic cell or precursor thereof, e.g., stem or progenitor cell, can be from any mammalian species, with non-limiting examples including a murine, bovine, simian, porcine, equine, ovine, or human cell. For clarity and simplicity, the description of the methods herein refers to a mammalian hypoimmunogenic cell or precursor thereof but it should be understood that all of the methods described herein can be readily applied to other cell types of hypoimmunogenic cells or precursors thereof. In some embodiments, the hypoimmunogenic cells or precursors thereof are derived from a human individual.

In some embodiments, the hypoimmunogenic cells or precursors thereof are a substantially pure population of hypoimmunogenic cells or precursors thereof. In one embodiment, the hypoimmunogenic cells or precursors thereof are a substantially pure population of hypoimmunogenic beta cells or precursors thereof. In some embodiments, a population of hypoimmunogenic cells or precursors thereof comprises a mixture of pluripotent cells or differentiated cells (e.g., a mixture of SC-cells and/or other differentiated cell types). In some embodiments, an SC-islet (e.g., a hypoimmunogenic SC-islet) comprises a mixture of pluripotent cells or differentiated cells (e.g., a mixture of SC-β cells and/or other differentiated cell types). In some embodiments, a population of hypoimmunogenic SC-islet cells or precursors thereof are substantially free or devoid of embryonic stem cells or pluripotent cells or iPS cells.

### Hypoimmunogenic Cells

In some aspects of the disclosure, hypoimmunogenic cells (e.g., hypoimmunogenic β cells) are provided. The hypoimmunogenic cells disclosed herein share many distinguishing features of native pancreatic cells but are different in certain aspects. In some embodiments, the hypoimmunogenic cells are non-native, i.e., non-naturally occurring, non-endogenous cells. As used herein, “non-native” means that the modified hypoimmunogenic cells are markedly different in certain aspects from cells which exist in nature, i.e., native cells. It should be appreciated, however, that these marked differences may result in the hypoimmunogenic cells exhibiting certain differences, but the hypoimmunogenic cells may still behave in a similar manner to native cells (e.g., with respect to the secretion of insulin in the case of beta cells) with certain functions altered (e.g., improved) compared to the native cells.

Hypoimmunogenic cells are differentiated in vitro from any starting cell, as the invention is not intended to be limited by the starting cell from which the hypoimmunogenic cells are derived. Exemplary starting cells include, without limitation, endocrine cells or any precursor thereof such as a NKX6-1+ pancreatic progenitor cell, a Pdxl+ pancreatic progenitor cell, and a pluripotent stem cell, an embryonic stem cell, and induced pluripotent stem cell. In some embodiments, the hypoimmunogenic cells are differentiated in vitro from a reprogrammed cell, a partially reprogrammed cell (i.e., a somatic cell, e.g., a fibroblast which has been partially reprogrammed such that it exists in an intermediate state between an induced pluripotency cell and the somatic cell from which it has been derived), a transdifferentiated cell. In some embodiments, the hypoimmunogenic cells disclosed herein can be differentiated in vitro from an endocrine cell or a precursor thereof. In some embodiments, the hypoimmunogenic cells is differentiated in vitro from a stem cell or progenitor cell, which may include a beta cell progenitor cell or a pluripotent stem cell. In some embodiments, the pluripotent stem cell is selected from the group consisting of an embryonic stem cell and induced pluripotent stem cell. In some embodiments, the hypoimmunogenic cells or the pluripotent stem cell from which the hypoimmunogenic cells is derived is human. In some embodiments, the hypoimmunogenic cell is human.

In some embodiments, the hypoimmunogenic cells (e.g., SC-beta cells) are modified to express or overexpress one or more tolerogenic transgenes from a locus in the stem cell or progenitor cell. Non-limiting examples of tolerogenic transgenes include PDL1, HLA-E/G, CD47, SERPINB9, CCL21, FASL, CD200, MFGE8, CD55, CD46, HLS-G single chain fusion, soluble PDL1-Ig and CTLA4-Ig. In one embodiment, the tolerogenic agent is HLA-E single chain fusion. The single chain fusion may be loaded with a peptide capable of activating NK cells, e.g., through interacting with the inhibitor receptor NKG2A and/or NKG2C. In some embodiments, the peptide may be VMAPRTLL (SEQ ID NO: 3), VMAPRTLFL (SEQ ID NO: 4), VMAPRTLVL (SEQ ID NO: 5), VMAPRTLIL (SEQ ID NO: 6), IMAPRTLVL (SEQ ID NO: 7), VMPPRTLLL (SEQ ID NO: 8), VMAPRTVLL (SEQ ID NO: 9), VTAPRTLLL (SEQ ID NO: 10), VTAPRTVLL (SEQ ID NO: 11), VMAPRTLTL (SEQ ID NO: 12) and/or VMAPRALLL (SEQ ID NO: 13).

In some embodiments, the hypoimmunogenic cells (e.g., SC-beta cells) are modified to express or overexpress one or more immunomodulatory agents from a locus in the stem cell or progenitor cell. In some embodiments, the immunomodulatory agent comprises a cytokine. In certain embodiments, immunomodulatory agents comprises IL-10, TGF-β, and/or IL-2. In one embodiment, the immunomodulatory agent is a cytokine. In one embodiment, the immunomodulatory agent is IL-10. In one embodiment, the immunomodulatory agent is TGF-β. In one embodiment, the immunomodulatory agent is IL-2.

In some embodiments, the locus is constitutively expressed in all cells of an islet, e.g., a human islet. In some embodiments, the locus is constitutively expressed in beta cells, e.g., in human beta cells. The locus may be the locus of a housekeeping gene. In some embodiments, the housekeeping gene includes actin, ubiquitin, and/or GAPDH. In one embodiment, the housekeeping gene is GAPDH.

Hypoimmunogenic cells may exhibit decreased risk of auto and/or allogeneic rejection upon transplant. In some aspects, the hypoimmunogenic cells secrete one or more immunomodulatory agents. For example, the hypoimmunogenic cells (e.g., hypoimmunogenic beta cells) secrete a cytokine, e.g., IL-10, TGF-β and IL-2 or a modified IL-2.

In some aspects, the disclosure provides a cell line comprising a hypoimmunogenic cell (e.g., hypoimmunogenic beta cells) described herein. In some aspects, the disclosure provides an SC-islet comprising hypoimmunogenic stem cell-derived β cells.

In some embodiments, the cells described herein, e.g., a population of hypoimmunogenic cells are transplantable, e.g., a population of hypoimmunogenic cells can be administered to a subject. In some embodiments, an SC-islet comprising hypoimmunogenic cells (e.g., hypoimmunogenic SC-beta cells) is transplantable, e.g., an SC-islet can be administered to a subject. In some embodiments, the subject who is administered a population of hypoimmunogenic cells is the same subject from whom a pluripotent stem cell used to differentiate into a hypoimmunogenic cell was obtained (e.g., for autologous cell therapy). In some embodiments, the subject is a different subject. In some embodiments, a subject is suffering from diabetes such as insulin-dependent diabetes or is a normal subject. For example, the cells for transplantation (e.g., a composition comprising a population of hypoimmunogenic cells or an SC-islet comprising a population of hypoimmunogenic cells) can be a form suitable for transplantation, e.g., organ transplantation.

The method can further include administering the cells to a subject in need thereof, e.g., a mammalian subject, e.g., a human subject. The source of the cells can be a mammal, preferably a human. The source or recipient of the cells can also be a non-human subject, e.g., an animal model. The term “mammal” includes organisms, which include mice, rats, cows, sheep, pigs, rabbits, goats, horses, monkeys, dogs, cats, and preferably humans. Likewise, transplantable cells can be obtained from any of these organisms, including a non-human transgenic organism. In one embodiment, the transplantable cells are genetically engineered, e.g., the cells include an exogenous gene or have been genetically engineered to inactivate or alter an endogenous gene.

A composition comprising a population of hypoimmunogenic cells (e.g., hypoimmunogenic pancreatic stem cell-derived cells, such as SC-β cells) can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

For administration to a subject, a cell population produced by the methods as disclosed herein, e.g., a population of hypoimmunogenic cells (e.g., hypoimmunogenic beta cells) or an SC-islet comprising hypoimmunogenic cells can be administered to a subject, for example in pharmaceutically acceptable compositions. These pharmaceutically acceptable compositions comprise a therapeutically effective amount of a population of hypoimmunogenic cells as described above, formulated together with one or more pharmaceutically acceptable carriers (additives) and/or diluents.

As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C2-C12 alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein in respect to a population of cells means that amount of relevant cells in a population of cells, e.g., hypoimmunogenic cells, or a composition comprising hypoimmunogenic cells of the present invention which is effective for producing some desired therapeutic effect in at least a sub-population of cells in an animal at a reasonable benefit/risk ratio applicable to any medical treatment. For example, an amount of a population of hypoimmunogenic cells administered to a subject that is sufficient to produce a statistically significant, measurable change in at least one symptom of Type 1, Type 1.5 or Type 2 diabetes, such as glycosylated hemoglobin level, fasting blood glucose level, hypoinsulinemia, etc. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

By “treatment,” “prevention,” or “amelioration” of a disease or disorder is meant delaying or preventing the onset of such a disease or disorder, reversing, alleviating, ameliorating, inhibiting, slowing down or stopping the progression, aggravation or deterioration of the progression or severity of a condition associated with such a disease or disorder. In one embodiment, the symptoms of a disease or disorder are alleviated by at least 5%, at least 10%, at least 20%, at least 30%, at least 40%, or at least 50%.

Treatment of diabetes is determined by standard medical methods. A goal of diabetes treatment is to bring sugar levels down to as close to normal as is safely possible. Commonly set goals are 80-120 milligrams per deciliter (mg/dl) before meals and 100-140 mg/dl at bedtime. A particular physician may set different targets for the patient, depending on other factors, such as how often the patient has low blood sugar reactions. Useful medical tests include tests on the patient's blood and urine to determine blood sugar level, tests for glycosylated hemoglobin level (HbA1c; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 1998). A successful treatment program can also be determined by having fewer patients in the program with complications relating to diabetes, such as diseases of the eye, kidney disease, or nerve disease.

Delaying the onset of diabetes in a subject refers to delay of onset of at least one symptom of diabetes, e.g., hyperglycemia, hypoinsulinemia, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

In certain embodiments, the subject is a mammal, e.g., a primate, e.g., a human. The terms, “patient” and “subject” are used interchangeably herein. Preferably, the subject is a mammal. The mammal can be a human, non-human primate, mouse, rat, dog, cat, horse, or cow, but are not limited to these examples. Mammals other than humans can be advantageously used as subjects that represent animal models of Type 1 diabetes, Type 2 Diabetes Mellitus, or pre-diabetic conditions. In addition, the methods described herein can be used to treat domesticated animals and/or pets. A subject can be male or female. A subject can be one who has been previously diagnosed with or identified as suffering from or having diabetes (e.g., Type 1 or Type 2), one or more complications related to diabetes, or a pre-diabetic condition, and optionally, but need not have already undergone treatment for diabetes, the one or more complications related to diabetes, or the pre-diabetic condition. A subject can also be one who is not suffering from diabetes or a pre-diabetic condition. A subject can also be one who has been diagnosed with or identified as suffering from diabetes, one or more complications related to diabetes, or a pre-diabetic condition, but who show improvements in known diabetes risk factors as a result of receiving one or more treatments for diabetes, one or more complications related to diabetes, or the pre-diabetic condition. Alternatively, a subject can also be one who has not been previously diagnosed as having diabetes, one or more complications related to diabetes, or a pre-diabetic condition. For example, a subject can be one who exhibits one or more risk factors for diabetes, complications related to diabetes, or a pre-diabetic condition, or a subject who does not exhibit diabetes risk factors, or a subject who is asymptomatic for diabetes, one or more diabetes-related complications, or a pre-diabetic condition. A subject can also be one who is suffering from or at risk of developing diabetes or a pre-diabetic condition. A subject can also be one who has been diagnosed with or identified as having one or more complications related to diabetes or a pre-diabetic condition as defined herein, or alternatively, a subject can be one who has not been previously diagnosed with or identified as having one or more complications related to diabetes or a pre-diabetic condition.

As used herein, the phrase “subject in need of hypoimmunogenic beta cells” refers to a subject who is diagnosed with or identified as suffering from, having or at risk for developing diabetes (e.g., Type 1, Type 1.5 or Type 2), one or more complications related to diabetes, or a pre-diabetic condition.

A subject in need of a population of hypoimmunogenic beta cells can be identified using any method used for diagnosis of diabetes. For example, Type 1 diabetes can be diagnosed using a glycosylated hemoglobin (A1C) test, a random blood glucose test and/or a fasting blood glucose test. Parameters for diagnosis of diabetes are known in the art and available to skilled artisan without much effort.

In some embodiments, the methods of the invention further comprise selecting a subject identified as being in need of additional hypoimmunogenic beta cells. A subject in need a population of hypoimmunogenic beta cells can be selected based on the symptoms presented, such as symptoms of type 1, type 1.5 or type 2 diabetes. Exemplary symptoms of diabetes include, but are not limited to, excessive thirst (polydipsia), frequent urination (polyuria), extreme hunger (polyphagia), extreme fatigue, weight loss, hyperglycemia, low levels of insulin, high blood sugar (e.g., sugar levels over 250 mg, over 300 mg), presence of ketones present in urine, fatigue, dry and/or itchy skin, blurred vision, slow healing cuts or sores, more infections than usual, numbness and tingling in feet, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, and combinations thereof.

In some embodiments, a composition comprising a population of hypoimmunogenic beta cells for administration to a subject can further comprise a pharmaceutically active agent, such as those agents known in the art for treatment of diabetes and or for having anti-hyperglycemic activities, for example, inhibitors of dipeptidyl peptidase 4 (DPP-4) (e.g., Alogliptin, Linagliptin, Saxagliptin, Sitagliptin, Vildagliptin, and Berberine), biguanides (e.g., Metformin, Buformin and Phenformin), peroxisome proliferator-activated receptor (PPAR) modulators such as thiazolidinediones (TZDs) (e.g., Pioglitazone, Rivoglitazone, Rosiglitazone and Troglitazone), dual PPAR agonists (e.g., Aleglitazar, Muraglitazar and Tesaglitazar), sulfonylureas (e.g., Acetohexamide, Carbutamide, Chlorpropamide, Gliclazide, Tolbutamide, Tolazamide, Glibenclamide (Glyburide), Glipizide, Gliquidone, Glyclopyramide, and Glimepiride), meglitinides (“glinides”) (e.g., Nateglinide, Repaglinide and Mitiglinide), glucagon-like peptide-1 (GLP-1) and analogs (e.g., Exendin-4, Exenatide, Liraglutide, Albiglutide), insulin and insulin analogs (e.g., Insulin lispro, Insulin aspart, Insluin glulisine, Insulin glargine, Insulin detemir, Exubera and NPH insulin), alpha-glucosidase inhibitors (e.g., Acarbose, Miglitol and Voglibose), amylin analogs (e.g. Pramlintide), Sodium-dependent glucose cotransporter T2 (SGLT T2) inhibitors (e.g., Dapgliflozin, Remogliflozin and Sergliflozin) and others (e.g. Benfluorex and Tolrestat).

In some aspects, a composition comprising a population of hypoimmunogenic beta cells for administration to a subject can further comprise a pharmaceutically active agent, such as those agents known in the art for suppressing the immune system, i.e., immunosuppressants. Non-limiting examples of immunosuppressants include biologics (e.g., adalimumab and infliximab), calcineurin inhibitors (e.g., tacrolimus and cyclosporine), corticosteroids (e.g., prednisone), inosine monophosphate dehydrogenase (IMDH) inhibitors (e.g., mycophenolate mofetil), janus kinase inhibitors (e.g., tofacitinib), mechanistic target of rapamycin (mTOR) inhibitors (e.g., sirolimus), and monoclonal antibodies (e.g., basiliximab).

A composition comprising hypoimmunogenic cells can be administrated to the subject at the same time or at different times as the administration of a pharmaceutically active agent or composition comprising the same. When administrated at different times, the compositions comprising a population of hypoimmunogenic cells and/or pharmaceutically active agent for administration to a subject can be administered within 5 minutes, 10 minutes, 20 minutes, 60 minutes, 2 hours, 3 hours, 4, hours, 8 hours, 12 hours, 24 hours of administration of the other. When a composition comprising a population of hypoimmunogenic cells and a composition comprising a pharmaceutically active agent are administered in different pharmaceutical compositions, routes of administration can be different. In some embodiments, a subject is administered a composition comprising hypoimmunogenic cells. In other embodiments, a subject is administered a composition comprising a pharmaceutically active agent. In another embodiment, a subject is administered a composition comprising a population of hypoimmunogenic cells mixed with a pharmaceutically active agent. In another embodiment, a subject is administered a composition comprising a population of hypoimmunogenic cells and a composition comprising a pharmaceutically active agent, where administration is substantially at the same time, or subsequent to each other.

Toxicity and therapeutic efficacy of administration of a composition comprising a population of hypoimmunogenic cells can be determined by standard pharmaceutical procedures in cell cultures or experimental animals, e.g., for determining the LD50 (the dose lethal to 50% of the population) and the ED50 (the dose therapeutically effective in 50% of the population). Compositions comprising a population of hypoimmunogenic cells that exhibit large therapeutic indices are preferred.

The amount of a composition comprising a population of hypoimmunogenic cells can be tested using several well-established animal models.

The non-obese diabetic (NOD) mouse carries a genetic defect that results in insulitis showing at several weeks of age (Yoshida et al., Rev. Immunogenet. 2:140, 2000). 60-90% of the females develop overt diabetes by 20-30 weeks. The immune-related pathology appears to be similar to that in human Type I diabetes. Other models of Type I diabetes are mice with transgene and knockout mutations (Wong et al., Immunol. Rev. 169:93, 1999). A rat model for spontaneous Type I diabetes was recently reported by Lenzen et al. (Diabetologia 44:1189, 2001). Hyperglycemia can also be induced in mice (>500 mg glucose/dL) by way of a single intraperitoneal injection of streptozotocin (Soria et al., Diabetes 49:157, 2000), or by sequential low doses of streptozotocin (Ito et al., Environ. Toxicol. Pharmacol. 9:71, 2001). To test the efficacy of implanted islet cells, the mice are monitored for return of glucose to normal levels (<200 mg/dL).

Larger animals provide a good model for following the sequelae of chronic hyperglycemia. Dogs can be rendered insulin-dependent by removing the pancreas (J. Endocrinol. 158:49, 2001), or by feeding galactose (Kador et al., Arch. Opthalmol. 113:352, 1995). There is also an inherited model for Type I diabetes in keeshond dogs (Am. J. Pathol. 105:194, 1981). Early work with a dog model (Banting et al., Can. Med. Assoc. J. 22:141, 1922) resulted in a couple of Canadians making a long ocean journey to Stockholm in February of 1925.

In some embodiments, data obtained from the cell culture assays and in animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose of a composition comprising a population of hypoimmunogenic cells can also be estimated initially from cell culture assays. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the stem cell-derived cells. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In another aspect of the invention, the methods provide use of an isolated population of hypoimmunogenic cells as disclosed herein. In one embodiment of the invention, an isolated population of hypoimmunogenic cells as disclosed herein may be used for the production of a pharmaceutical composition, for use in transplantation into subjects in need of treatment, e.g., a subject that has, or is at risk of developing diabetes, for example but not limited to subjects with congenital and acquired diabetes. In one embodiment, an isolated population of hypoimmunogenic cells may be genetically modified. In another aspect, the subject may have or be at risk of diabetes and/or metabolic disorder. In some embodiments, an isolated population of hypoimmunogenic cells as disclosed herein may be autologous and/or allogeneic. In some embodiments, the subject is a mammal, and in other embodiments the mammal is a human.

The use of an isolated population of hypoimmunogenic cells as disclosed herein provides advantages over existing methods because the population of hypoimmunogenic cells can be differentiated from endocrine progenitor cells or precursors thereof derived from stem cells, e.g., iPS cells obtained or harvested from the subject administered an isolated population of hypoimmunogenic cells. This is highly advantageous as it provides a renewable source of hypoimmunogenic cells which can be differentiated from stem cells to endocrine progenitor cells by methods commonly known by one of ordinary skill in the art, and then further differentiated by the methods described herein to pancreatic 3-like cells for transplantation into a subject, in particular an SC-islet comprising hypoimmunogenic pancreatic endocrine cells that do not have the risks and limitations of cells derived from other systems.

One embodiment of the invention relates to a method of treating diabetes or a metabolic disorder in a subject comprising administering an effective amount of a composition comprising a population of hypoimmunogenic cells (e.g., hypoimmunogenic beta cells) as disclosed herein to a subject with diabetes and/or a metabolic disorder. In a further embodiment, the invention provides a method for treating diabetes, comprising administering a composition comprising a population of hypoimmunogenic cells as disclosed herein to a subject that has, or has an increased risk of developing diabetes.

In one embodiment of the above methods, the subject is a human and a population of hypoimmunogenic cells as disclosed herein are human cells. In some embodiments, the invention contemplates that a population of hypoimmunogenic cells as disclosed herein are administered directly to the pancreas of a subject or is administered systemically. In some embodiments, a population of hypoimmunogenic cells as disclosed herein can be administered to any suitable location in the subject, for example in a capsule in the blood vessel or the liver.

The present invention is also directed to a method of treating a subject with diabetes or a metabolic disorder which occurs as a consequence of genetic defect, physical injury, environmental insult or conditioning, bad health, obesity and other diabetes risk factors commonly known by a person of ordinary skill in the art. Efficacy of treatment of a subject administered a composition comprising a population of hypoimmunogenic cells (e.g., hypoimmunogenic beta cells) can be monitored by clinically accepted criteria and tests, which include for example, (i) Glycated hemoglobin (A1C) test, which indicates a subjects average blood sugar level for the past two to three months, by measuring the percentage of blood sugar attached to hemoglobin, the oxygen-carrying protein in red blood cells. The higher your blood sugar levels, the more hemoglobin has sugar attached. An A1C level of 6.5 percent or higher on two separate tests indicates the subject has diabetes. A test value of 6-6.5% suggest the subject has prediabetes. (ii) Random blood sugar test. A blood sample will be taken from the subject at a random time, and a random blood sugar level of 200 milligrams per deciliter (mg/dL)-11.1 millimoles per liter (mmol/L), or higher indicated the subject has diabetes. (iii) Fasting blood sugar test. A blood sample is taken from the subject after an overnight fast. A fasting blood sugar level between 70 and 99 mg/dL (3.9 and 5.5 mmol/L) is normal. If the subjects fasting blood sugar levels is 126 mg/dL (7 mmol/L) or higher on two separate tests, the subject has diabetes. A blood sugar level from 100 to 125 mg/dL (5.6 to 6.9 mmol/L) indicates the subject has prediabetes. (iv) Oral glucose tolerance test. A blood sample will be taken after the subject has fasted for at least eight hours or overnight and then ingested a sugary solution, and the blood sugar level will be measured two hours later. A blood sugar level less than 140 mg/dL (7.8 mmol/L) is normal. A blood sugar level from 140 to 199 mg/dL (7.8 to 11 mmol/L) is considered prediabetes. This is sometimes referred to as impaired glucose tolerance (IGT). A blood sugar level of 200 mg/dL (11.1 mmol/L) or higher may indicate diabetes.

In some embodiments, the effects of administration of a population of hypoimmunogenic cells (e.g., hypoimmunogenic beta cells) as disclosed herein to a subject in need thereof is associated with improved exercise tolerance or other quality of life measures, and decreased mortality. The effects of cellular therapy with a population of hypoimmunogenic cells can be evident over the course of days to weeks after the procedure. However, beneficial effects may be observed as early as several hours after the procedure and may persist for several years. In some embodiments, the effects of cellular therapy with a population of hypoimmunogenic cells occurs within two weeks after the procedure.

In some embodiments, a population of hypoimmunogenic cells (e.g., hypoimmunogenic beta cells) as disclosed herein may be used for tissue reconstitution or regeneration in a human patient or other subject in need of such treatment. In some embodiments compositions of populations of hypoimmunogenic cells can be administered in a manner that permits them to graft or migrate to the intended tissue site and reconstitute or regenerate the functionally deficient area. Special devices are available that are adapted for administering cells capable of reconstituting a population of hypoimmunogenic pancreatic cells (e.g., β cells) in the pancreas or at an alternative desired location. Accordingly, the hypoimmunogenic cells may be administered to a recipient subject's pancreas by injection or administered by intramuscular injection.

### Examples

Persistent transgene expression is essential for maintaining the desired hypoimmunogenic phenotype both throughout differentiation and post transplantation. Previously, transgene silencing was observed when targeting the expression of tolerogenic molecules to the AAVS1 safe-harbor locus in hESCs followed by differentiation into SC-beta cells. Since housekeeping genes such as GAPDH are constitutively expressed in all cells of the human islet, it was hypothesized that targeting the expression of tolerogenic molecules at the GAPDH locus in hESCs would result in strong and persistent transgene expression following SC-islet cell differentiation and post transplantation. Table 1 provides a non-exhaustive list of other housekeeping genes suitable for use in the invention.

| | Gene |
|---|---|
| Housekeeping Gene | symbol |
| 18S ribosomal RNA | RRN18S |
| Actin, beta | ACTB |
| Glyceraldehyde-3-phosphate dehydrogenase | GAPDH |
| Phosphoglycerate kinase 1 | PGK1 |
| Peptidylprolyl isomerase A | PPIA |
| Ribosomal protein L13a | RPL13A |
| Ribosomal protein, large, P0 | RPLP0 |
| Acidic ribosomal phosphoprotein P0 | ARBP |
| Beta-2-microglobulin | B2M |
| Tyrosine | YWHAZ |
| 3-monooxygenase/tryptophan5-monooxygenase | |
| activation protein, zeta polypeptide | |
| Succinate dehydrogenase complex, subunit A, | SDHA |
| flavoprotein (Fp) | |
| Transferrin receptor | TFRC |
| Glucuronidase, beta | GUSB |
| Hydroxymethylbilane synthase | HMBS |
| Hypoxanthine phosphoribosyltransferase 1 | HPRT1 |
| TATA box binding protein | TBP |

### Generation of Hypoimmunogenic SC-Islet Cells

A targeted approach was taken using the least number of editing steps to engineer hypoimmunogenic SC-islet cells. First, we interfered with the ability of stem cells and their differentiated progeny to present antigen by eliminating HLA class I expression through knockout of the B2M gene in hESCs. In doing so, we reduce the ability of T cells to engage and destroy allogeneic SC-islet cells. We have also targeted the expression of the immune-regulatory molecule PDL1 at the GAPDH locus to provide additional inhibitory signaling against T cells. Other candidate molecules for expression from the GAPDH locus include soluble PDL1-Ig and CTLA4-Ig, CD47 (macrophage and NK cell inhibition), HLA-G single chain fusion (NK cell inhibition), SERPINB9 (granzyme inhibitor), CCL21 (macrophage cytokine inhibitor), FASL (T cell apoptosis), CD200 (myeloid cell inhibition), CD55 and CD46 (complement inactivation). In the case of multiple transgene expression, a polycistronic RNA is transcribed from the GADPH locus encoding GAPDH and the tolerogenic molecules separated by self-cleaving peptides. We chose not to interfere with HLA class II expression since its expression has not been observed in our SC-islet cells.

Since HLA class I deficient cells are susceptible to NK cell cytotoxicity, we then introduced the HLA-E single chain fusion gene at the GAPDH locus such that SC-islet cells would express a single HLA molecule that is involved in NK cell inhibition. Additionally, we have selectively loaded a peptide VMAPRTLLL that has been shown to result in the preferential interaction between HLA-E and its cognate inhibitory receptor NKG2A on NK cells12. By comparison, other HLA-E single chain fusions loaded with a HLA-G leader sequence-derived peptide (VMAPRTLFL)3 have been shown to activate NK cells through their interaction with the HLA-E cognate activating receptor NKG2C. Other potential candidate peptides for loading into the HLA-E fusion that do not activate NK cells through NKG2C include VMAPRTLVL, VMAPRTLIL, IMAPRTLVL, VMPPRTLLL, VMAPRTVLL, VTAPRTLLL, VTAPRTVLL, VMAPRTLTL and VMAPRALLL. Following the generation of hypoimmunogenic hESCs, the cells are differentiated into SC-β cells that lack classical HLA expression and constitutively express tolerogenic molecules that are capable of inhibiting T cell and NK cell responses in an allogeneic context.

### SC-Islet Cells as Mediators of Localized Immune-Tolerance

We also sought to use our engineering strategy to provide SC-islet cells the ability to modulate their local immune microenvironment via the secretion of immune-modulatory cytokines. By doing so, we hypothesized that immune-modulatory cytokines secreted by islet cells could induce localized immune-tolerance, thereby protecting the allograft from immune destruction. We modified the GAPDH locus-targeting constructs to contain the tolerogenic cytokines IL-10, TGF-3 and a modified IL-2, and introduced the cytokines into the GAPDH locus of hESCs. Thereafter, cytokine-secreting hESCs were differentiated into SC-islet cells and transplanted into immune-competent mice.

### Methods

### Mice

Male B6/albino, Scid/beige and NSG-MHC Class I/II KO mice (6-8 weeks of age) were purchased from Jackson Labs. Mice were housed in specific pathogen-free conditions at Harvard University. All animal research was conducted under Harvard IACUC approval.

### Cell Culture

Human ES maintenance and differentiation was carried out as previously described (Millman et al., 2016; Pagliuca et al., 2014). SC-islet cell differentiations were initiated 72 h after initial passage by aspirating mTeSR1 and replenished with stage and day-specific media supplemented with the appropriate small molecules or growth factors as previously described (Millman et al., 2016; Pagliuca et al., 2014; Veres et al., 2019). All cell lines were routinely tested for mycoplasma and were mycoplasma-free. All experiments involving human cells were approved by the Harvard University IRB and ESCRO committees.

Generation of Immune-Evasive hESCs

The existing GAPluc (WT) hESC line served as starting material for the generation of HLA-deficient hESCs (Gerace et al., 2021). To generate HLA-deficient hESCs, 1×106 WT hESCs were nucleofected using the 4D-Nucleofector (Lonza) with RNP complexed with 120 pmol B2M gRNA (5′-GCTACTCTCTCTTTCTGGCC′3; SEQ ID NO: 1)(Mandal et al., 2014) (IDT) and 104 pmol Alt-R® S.p. HiFi Cas9 Nuclease V3 (IDT) according to the manufacturer's instructions. Nucleofected cells were resuspended in mTesR1 (STEMCELL Technologies, 85850)+10 μM Y27632 (DNSK International, DNSK-KI-15-02) and plated in a matrigel-coated tissue culture plate. After 48 h cells were treated with 10 ng/ml IFN-γ (R&D, 285-IF-100) for 24 h and then stained with APC anti-human HLA-ABC (W6/32, 1:100) (Biolegend, 311409). HLA-ABC−/− cells were sorted on a FACS Aria II (BD Biosciences) and plated in a matrigel-coated tissue culture plate containing mTesR1+CloneR (STEMCELL Technologies, 05888), with single colonies picked for expansion. The resulting HLA-deficient line was named B2M−/−.

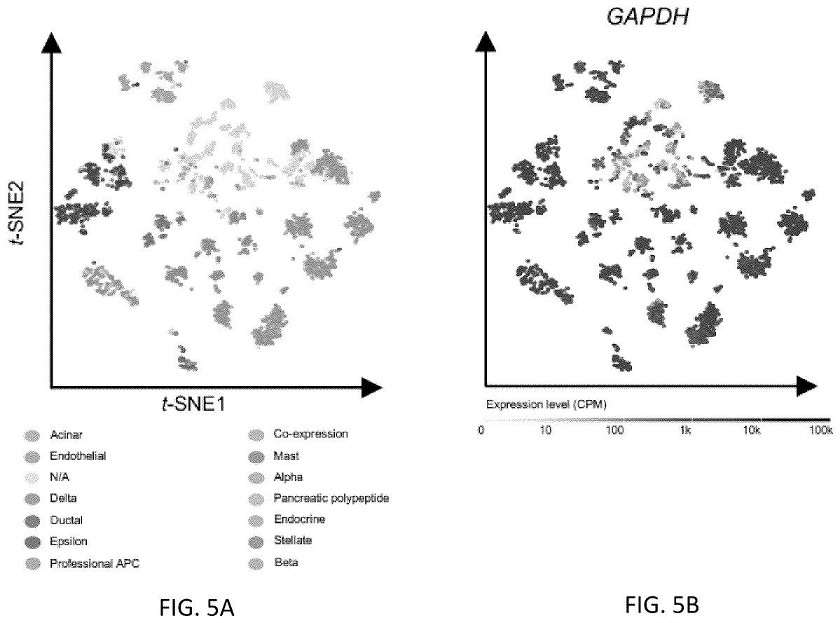

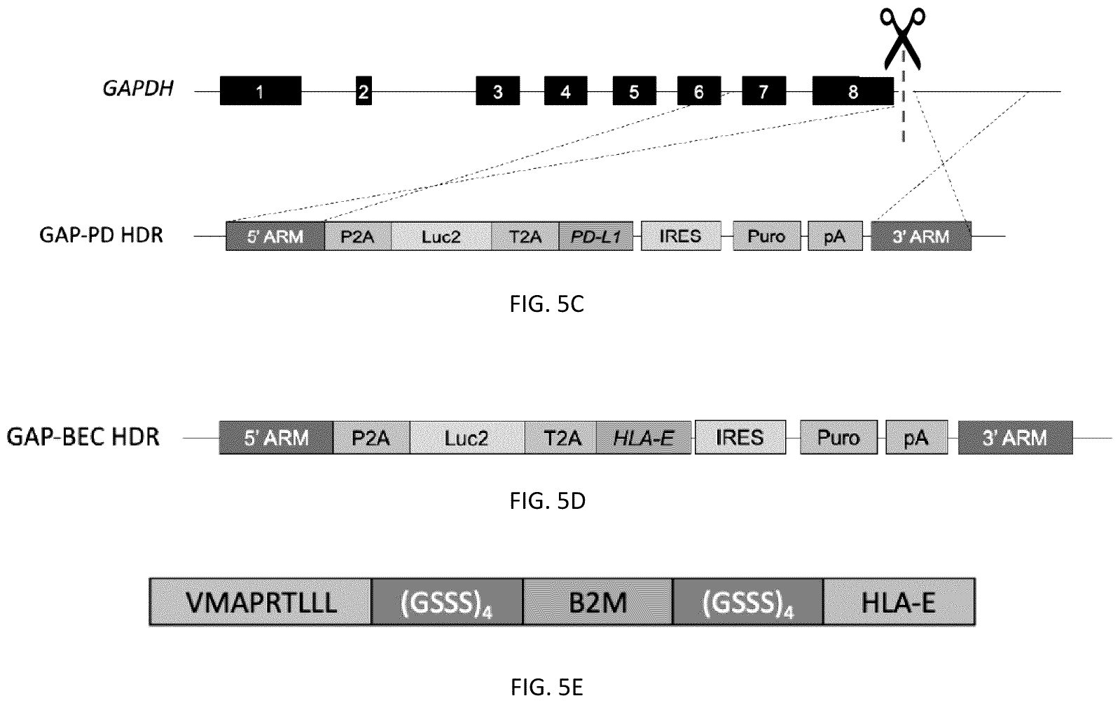

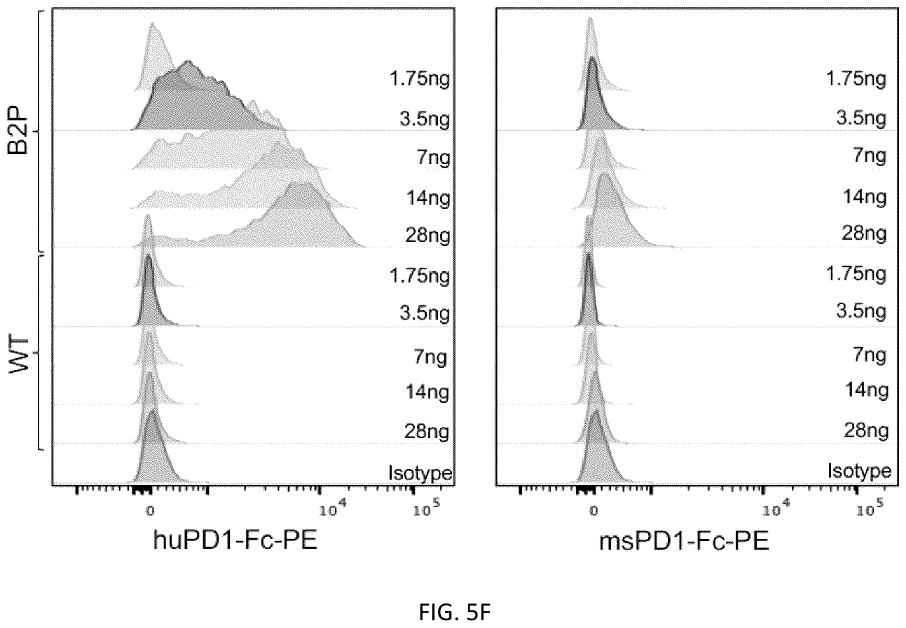

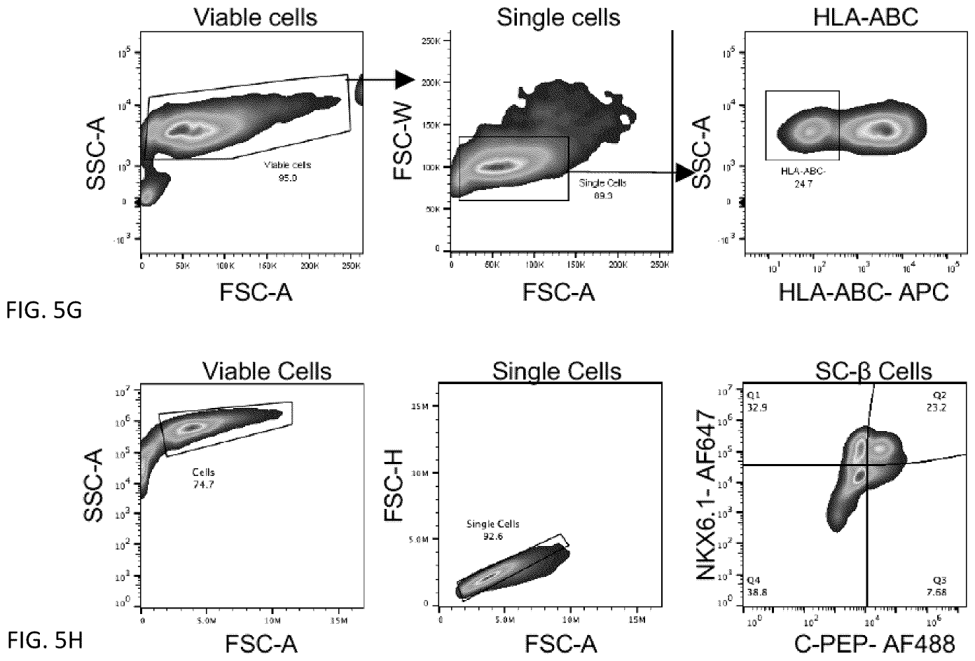

To generate GAP-PD and GAP-BEC hESCs, human PD-L1 and the peptide::B2M::HLA-E fusion sequences were synthesized as gBlocks (Genscript) and cloned into our existing GAPluc targeting plasmid downstream of the Luc2 gene. The sequence of the covalently attached peptide (VMAPRTLLL (SEQ ID NO: 2)) in the peptide::B2M::HLA-E fusion is derived from the HLA-Cw7 molecule (Kaiser et al., 2005). hESCs were nucleofected with the GAP-PD or GAP-BEC targeting plasmids and GAPDH-targeting RNP as previously described (Gerace et al., 2021). For GAP-B2P and GAP-BEC lines, a polyclonal population of puromycin-selected cells was subsequently nucleofected with B2M-targeting RNP, and single-colonies were picked for expansion after FACS sorting as described above. The gating strategy for sorting HLA-ABC−/− hESCs is described in FIG. 5G.

### Flow Cytometry

Differentiated WT, B2M−/−, BEC, PD-L1 and B2P SC-islet cells were both treated and untreated with 10 ng/ml IFN-γ for 24 h prior to staining. Cells were dissociated with Accutase (STEMCELL Technologies, 07920), washed twice with PBS+0.1% BSA (Gibco, A10008-01) and blocked for 30 min on ice with PBS+5% donkey serum (Jackson Labs; 100181-234). Cells were then stained for 30 min on ice in blocking buffer with PE or APC anti-human HLA-ABC (Biolegend, 311405), PE anti-human HLA-E (Biolegend, 342603) and PE anti-human PD-L1 (Biolegend, 393607). Cells were then washed three times and fixed in 4% PFA (EMS, 15710) for 15 min at 4° C. Fixed cells were then incubated in blocking buffer with rat anti-human C-peptide (DHSB; GN-ID4) and mouse anti-human Nk×6.1 (DHSB; F55A12) (overnight at 4° C.), washed three times with blocking buffer, incubated with goat anti-rat 647 (Life Technologies, A-21247; 1:300) and goat anti-mouse 405 (Life Technologies, A-31553; 1:300) in blocking solution (1 h at room temperature), washed three times and resuspended in PBS+0.1% BSA. Samples were captured on the LSR II (BD) flow cytometer and analyzed using FlowJo 10.7.1 (BD). All antibodies were used at 1:100 unless otherwise stated. The gating strategy for identifying SC-β cells is described in FIG. 5H. PE mouse IgG2a (Biolegend, 400213) and PE mouse IgG1 (Biolegend, 400111) served as isotype controls.

### Magnetic Enrichment Using CD49a and Reaggregation

WT and BEC SC-β cells were magnetically-enriched from SC-islet clusters as previously described (Veres et al., 2019). Enriched cells were resuspended in S6 medium and plated at 5×103 cells/well in low-attachment 96-well v-bottom tissue-culture plates (Thermo Scientific, 277143), centrifuged at 300 g for 1 min and incubated at 37° C. for 4-7 days. CD49a enriched SC-β cell clusters were fed fresh S6 medium every 2 days.

### SC-β Cell Immunohistochemistry

CD49a enriched SC-β cell clusters were fixed in 4% PFA for 1 h at room temperature, washed and frozen in OCT (Tissue-Tek, 4583) and sectioned to 14 μm. Before staining, paraffin-embedded samples were treated with Histo-Clear (EMS, 64110-01) to remove the paraffin. For staining, slides were incubated in blocking buffer (PBS+0.1% Triton-X+5% donkey serum) for 1 h at room temperature, incubated in PBS+5% donkey serum containing rat anti-human C-peptide (1:300), mouse anti-human Nk×6.1 (1:100) and rabbit anti-human HLA-E (Sigma-Aldrich, HPA031454, 1:100) for 1 h at room temperature, washed three times, incubated in goat anti-mouse 594 (Life Technologies, A-11032; 1:500), goat anti-rat 488 (Life Technologies, A-11006; 1:500) and goat anti-rabbit 647 (Life technologies, A-21244; 1:500) for 2 h at room temperature, washed, mounted in Vectashield with DAPI (Vector Laboratories; H-1200), covered with coverslips and sealed with clear nail polish. Representative regions were imaged using Zeiss.Z2 with Apotome microscope.

### Immune Cell Isolation

PBMCs, CD8 and CD4 T cells, and NK cells were isolated from apheresis leukoreduction collars (n=5 donors) obtained from Brigham and Women's Hospital in compliance with IRB approval. PBMCs were isolated by density gradient centrifugation with lymphoprep (STEMCELL Technologies, 07801) SepMate™-50 (IVD) tubes (STEMCELL Technologies, 85450) according to the manufacturer's instructions. CD4, CD8 and NK cells were isolated by density gradient centrifugation with lymphoprep and SepMate™-50 (IVD) tubes following the addition of RosetteSep Human CD4+ T Cell Enrichment Cocktail (STEMCELL Technologies, 15062), CD8+ T Cell Enrichment Cocktail (STEMCELL Technologies, 15063) and NK Cell Enrichment Cocktail (STEMCELL Technologies, 15065) respectively. Enriched immune cells were cryopreserved in CryoStor CS10 (STEMCELL Technologies, 07930) at a concentration of 10M cells/vial.

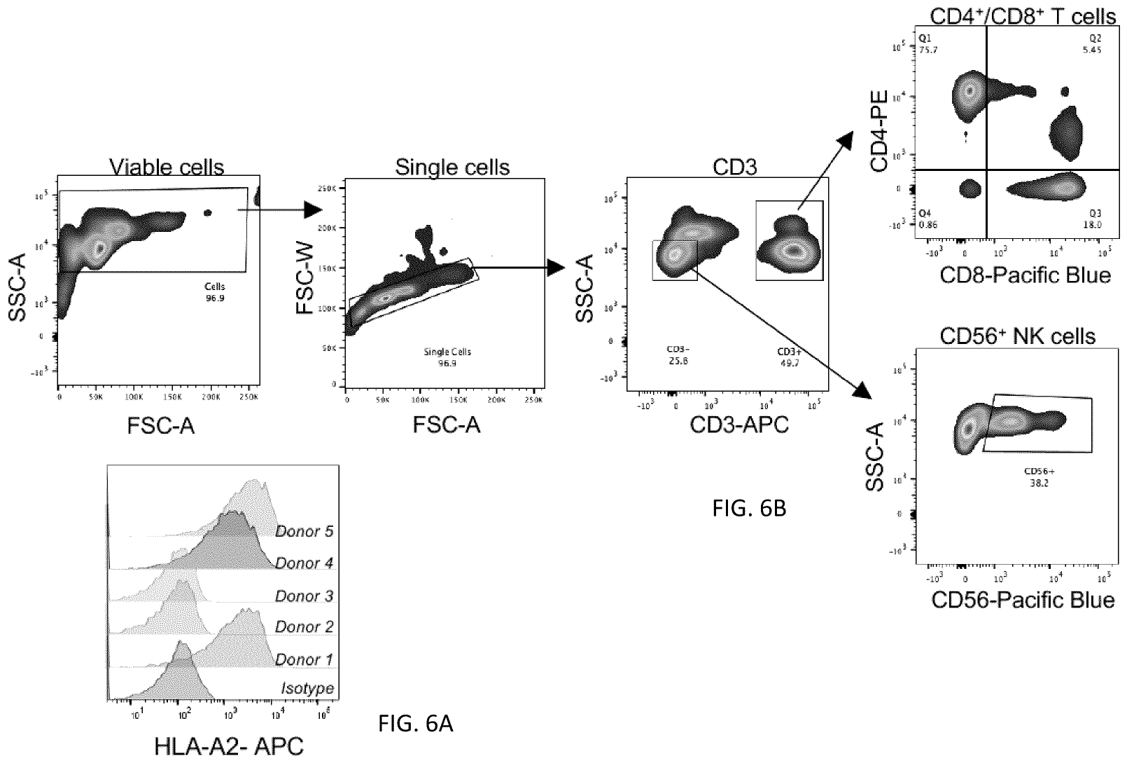

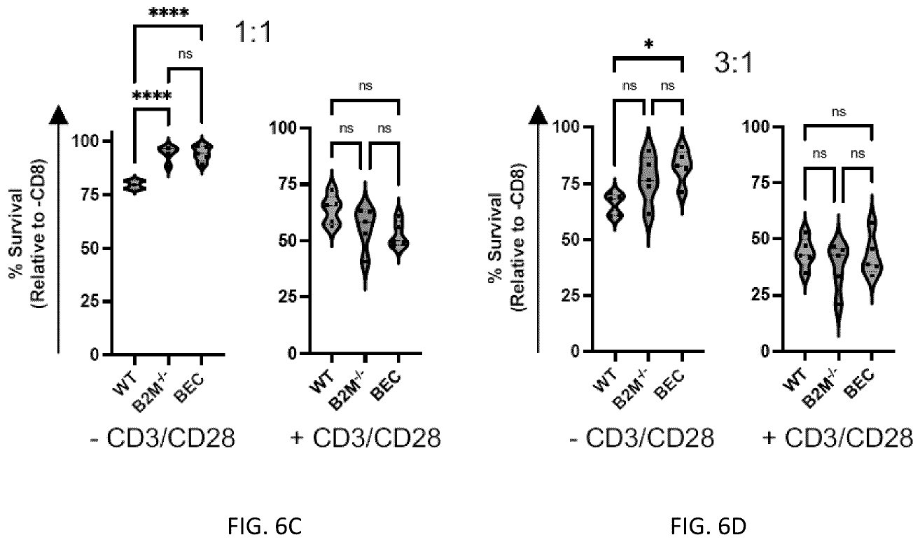

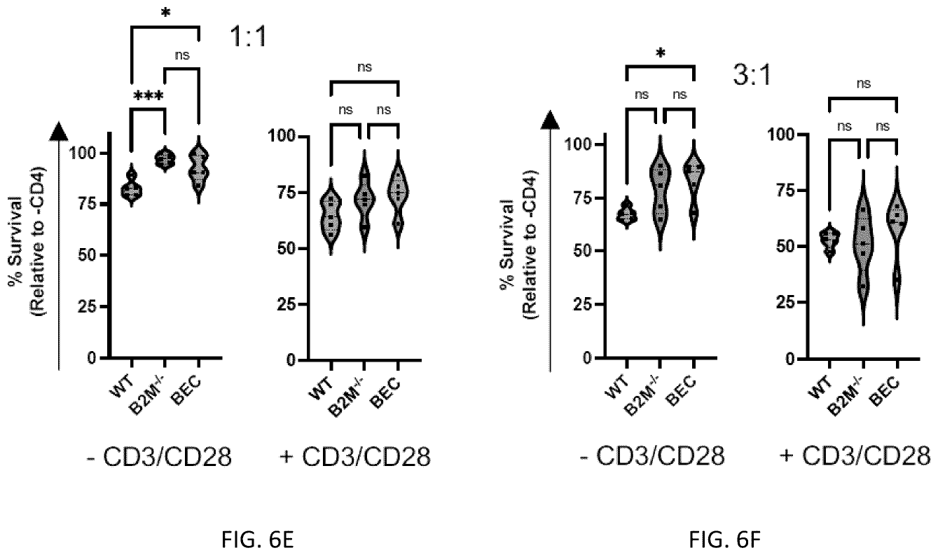

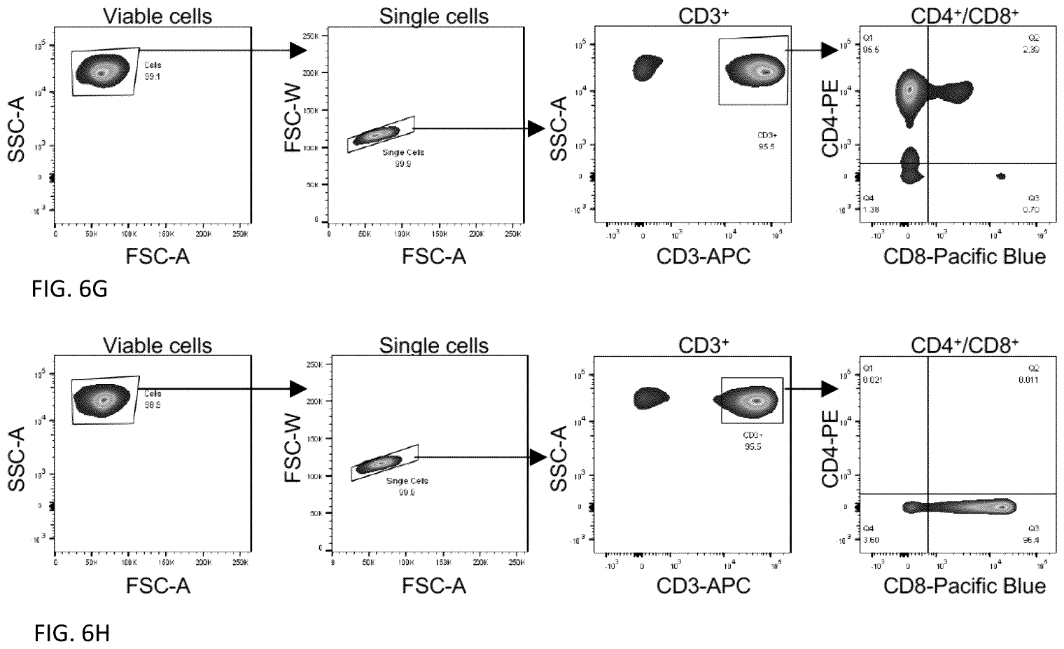

Flow cytometric analysis of enriched immune cell subpopulations was performed by staining cells in blocking buffer containing APC anti-human CD3 (Biolegend, 300311), PE anti-human CD4 (Biolegend, 357403), Pacific Blue anti-human CD8 (Biolegend, 344717) and Pacific Blue anti-human CD56 (Biolegend, 362519). Isotype controls were Pacific Blue mouse IgG1 (Biolegend, 400131), APC mouse IgG2a (Biolegend, 400221) and PE rat IgG2b (Biolegend, 400607). All antibodies were used at 1:100 or unless otherwise stated. Representative gating strategies are demonstrated in FIGS. 6G-6I.

### In Vitro T Cell Cytotoxicity Assays

WT, B2M−/− and BEC SC-islet cells were seeded in S3 medium at 5×104 cells/well of a matrigel-coated 96-well black flat-bottom plate (Corning, 3916) in the presence or absence of IFN-γ (10 ng/ml). After 24 h, the medium was switched to T cell medium (ImmunoCult™-XF T Cell Expansion Medium (STEMCELL Technologies, 10981)+100U/ml rhIL-2) for immune cell co-culture. Primary human PBMCs, CD4 or CD8 cells (n=5 donors) cultured in T cell medium were added to SC-islet cells at 1:1 and 3:1 effector:target ratios with and without the addition of ImmunoCult™ Human CD3/CD28 T Cell Activator (STEMCELL Technologies, 10991). All T cell cytotoxicity assays were co-cultured for 72 h, after which luminescence was measured following the addition of 150 μg/ml D-luciferin (Gold Biotechnology, LUCK-2G) on the CLARIOstar microplate reader (BMG LABTECH). SC-islet cell survival was calculated as a percentage relative to luminescence in the absence of T cells (test SC-islet cell luminescence/no T cell SC-islet cell luminescence×100).

### In Vitro NK Cell Cytotoxicity Assays

WT, B2M−/− and BEC SC-islet cells were seeded in NK cell medium (NK MACS medium (Milteny Biotec, 130-114-429)+5% Human AB serum (Valley Biomed, HP1022HI)+5% HyClone FBS (GE Healthcare, SH30070.03)+0.5 ng/ml rhIL-2 (Peprotech, 200-02)) at 2×104 cells/well of a 96-well black round-bottom ultra-low attachment plate (Corning, 4591). Primary NK cells (n=5 donors) cultured in NK cell media for 5 days were added to SC-islet cells at 1:1 and 10:1 effector:target ratios. K562-Luc2 (Biocytogen, BCG-PS-015-luc) and Raji-GFP-Luc2 (Biocytogen, BCG-PS-087-luc) cells were used as positive and negative controls respectively. All NK cell cytotoxicity assays were co-cultured for 5 h, after which luminescence and SC-islet cell survival were measured as described above.

### In Vivo NK Cell Cytotoxicity

2.5×105 WT and B2M−/− SC-islet cell clusters were resuspended either alone or with 7.5×105 primary human NK cells (pre-treated with 0.5 ng/ml rhIL-2 for 12 h) in phenol-free Matrigel (Corning, 356237) and transplanted subcutaneously in Scid/beige mice (n=5). Bioluminescence was measured 10 min after i.p injection of 10 μL/g D-luciferin (15 mg/ml) on days 1 and 5 following cell transplantation on the IVIS Spectrum (PerkinElmer, 124262) as previously described (Gerace et al., 2021).

### Endothelial Cell Differentiation

WT and B2M−/− hESCs were seeded in mTesR1+10 μM Y27632 at a concentration of 7.5×10⁴ cells/well of a Matrigel-coated 6-well tissue-culture plate (Corning, 3516). Thereafter, cells were differentiated into endothelial cells using STEMdiff™ Endothelial Differentiation Kit (STEMCELL Technologies, 08005) according to the manufacturer's instructions. Differentiation efficiency was quantified by FACS analysis after staining endothelial cells with Pacific Blue anti-human CD31 (Biologend, 303113). Pacific Blue mouse IgG1 (Biolegend, 400131) was used as an isotype control. All antibodies were used at 1:100 unless otherwise stated.

### Bulk RNA Sequencing

Magnetically-enriched CD49a+SC-β cells and SC-endothelial cells were seeded in 6-well plates at 1×10⁶ cells/well (in triplicate) and treated with and without 10 ng/ml IFN-γ for 24 h. Cells were then harvested using Accutase and the pellets snap-frozen on dry-ice. RNA extractions, library preparations and sequencing reactions were conducted at GENEWIZ, LLC. (South Plainfield, NJ, USA). Total RNA was extracted from cell pellet samples using RNeasy Plus Mini Kit (Qiagen, Germantown, MD, USA) and quantified using Qubit Fluorometer (Life Technologies, Carlsbad, CA, USA). SMART-Seq v4 Ultra Low Input Kit for Sequencing was used for full-length cDNA synthesis and amplification (Clontech, Mountain View, CA), and Illumina Nextera XT library was used for sequencing library preparation.

Multiplexed sequencing libraries were loaded on the Illumina HiSeq instrument and sequenced using a 2×150 Paired End (PE) configuration. Image analysis and base calling were conducted by the HiSeq Control Software (HCS). Raw sequence data (.bcl files) generated from Illumina HiSeq was converted into FASTQ files and de-multiplexed using Illumina's bcl2fastq 2.17 software. One mismatch was allowed for index sequence identification.

After investigating the quality of the raw data, sequence reads were trimmed to remove possible adapter sequences and nucleotides with poor quality using Trimmomatic v.0.36. The trimmed reads were mapped to the Homo sapiens reference genome available on ENSEMBL using the STAR aligner v.2.5.2b. The STAR aligner is a splice aligner that detects splice junctions and incorporates them to help align the entire read sequences. BAM files were generated as a result of this step. Unique gene hit counts were calculated by using feature Counts from the Subread package v.1.5.2. Only unique reads that fell within exon regions were counted.

After extraction of gene hit counts, the gene hit counts table was used for downstream differential expression analysis. Using DESeq2, a comparison of gene expression between the groups of samples was performed. The Wald test was used to generate p-values and Log 2 fold changes. Genes with adjusted p-values<0.05 and absolute log 2 fold changes>1 were called as differentially expressed genes for each comparison. A gene ontology analysis was performed on the statistically significant set of genes by implementing the software g:Profiler (Raudvere et al., 2019). The gene accession number for the unique RNAseq dataset generated in this study is GSE200021.

### NK Cell Ligand Analysis

WT and B2M−/− SC-islet cells and SC-endothelial cells were washed twice with washing buffer and blocked for 30 min on ice with blocking buffer. Cells were then stained for 30 min on ice in blocking buffer with APC anti-human CD47 (Biolegend, 323123), APC anti-human CD324 (E-Cadherin) (Biolegend, 324107), PE anti-human CD325 (N-Cadherin) (Biolegend, 350806), PE anti-human CD112 (Nectin-2) (Biolegend, 337409), APC anti-human CD155 (PVR) (Biolegend, 337617), PE anti-human CD111 (Nectin-1) (Biolegend, 340404), APC anti-human MICA/MICB (Biolegend, 320907), AF647 anti-human PCNA (Biolegend, 307912), PE anti-human BAG6 (Abcam, ab210838), APC anti-human CD48 (Biolegend, 336713), PE anti-human CD70 (Biolegend, 355103), mouse anti-human CD113 (Nectin-3) (Millipore-Sigma, MABT63), APC anti-human ULBP1 (R&D, FAB1380A), PE anti-human ULBP2/5/6 (R&D, FAB1298P), APC anti-human CLEC2D (R&D, FAB3480A) and PE anti-human CD72 (Biolegend, 316207). PE Mouse IgG1 (Biolegend, 400111), APC mouse IgG1 (Biolegend, 400121), APC mouse IgG2a (Biolegend, 400221), PE Mouse IgG2a (Biolegend, 400213), APC rat IgG1 (Biolegend, 401903), PE rabbit IgG (Cell Signaling technology, 5742S) and goat anti-mouse 488 (Thermofisher, A-11001, 1:300) served as isotype controls. All antibodies were used at 1:100 unless otherwise stated.

### Transplantation of HLA-Deficient SC-Islet Cells

Diabetes was induced in NSG-MHC Class I/II KO mice by multiple low-dose (40 mg/kg) streptozotocin (STZ) i.p. injection as previously described (Furman, 2021). Once animals reached a blood glucose of >500 mg/dL, 5×10⁶ WT or B2M−/− SC-islet cells were transplanted under the kidney capsule (n=10) as previously described (Millman et al., 2016; Pagliuca et al., 2014). Blood glucose and body weight was measured twice a week after transplantation.

Ten weeks after transplantation (before PBMC injection) and seven weeks after PBMC injection the function of transplanted cells was assessed by performing in vivo peritoneal glucose-stimulated insulin secretion (IPGTT) as previously described (Millman et al., 2016; Pagliuca et al., 2014). Insulin secretion was quantified using the Human Ultrasensitive Insulin ELISA (ALPCO Diagnostics; 80-INSHUU-E01.1.)

### Generation of Immune-Tolerizing SC-Islet Cells

The cDNAs of human IL-2, TGF-0 and IL-10 were synthesized as a polycistronic gBlock (Genscript) and cloned into the existing GAPluc targeting plasmid as described above to generate the GAP-2B10 plasmid. The human IL-2 sequence was modified by substituting a single amino acid (N88D) as previously described (Peterson et al., 2018). GAP-2B10 hESCs were generated by co-nucleofection of the GAP-2B10 targeting plasmid and the GAPDH-targeting RNP as described above. GAP-2B10 SC-islet cells were differentiated as previously described (Millman et al., 2016; Pagliuca et al., 2014).

### In Vitro Glucose-Stimulated Insulin Secretion

In vitro function was assessed by measuring glucose-stimulated insulin secretion (GSIS) as previously described (Millman et al., 2016; Pagliuca et al., 2014). SC-islet clusters were washed twice in Krebs buffer (KRB), and preincubated at 37° C. for 1 h in KRB containing 2 mM glucose (low glucose). Clusters were then challenged with three sequential treatments of alternating low-high-low KRB containing glucose (high; 20 mM), followed by depolarization with low KRB containing 30 mM KCl. Each treatment lasted 30 min, after which 100 μl of supernatant was collected and human insulin quantified using the Human Ultrasensitive Insulin ELISA. Human insulin measurements were normalized by viable cell counts that were acquired by dispersing clusters with TrypLE Express (Thermofisher, 12604013) and counted using a ViCell (Beckman Coulter).

### In Vitro SC-Islet Cell Cytokine Secretion and PBMC Cytotoxicity Assays

GAP-2B10 SC-islet cell clusters were dispersed with TrypLE Express and seeded in 96-well matrigel-coated plates in S3 media at a linear concentration (1, 2, 4, 6, 8×10⁴, and 1×10⁵ cells/well) in duplicate. After 24 h, supernatants were collected, centrifuged at 3000 g for 5 min and the cytokines IL-2, TGF-β and IL-10 quantified using Legend Max ELISAs (IL-2, Biolegend, 431807; TGF-β, Biolegend, 436707; IL-10, Biolegend, 430607) according to the manufacturer's instructions. For cytotoxicity assays, WT and 2B10 SC-islet cells were co-cultured with human PBMCs as described above.

### Xenotransplantation of GAP-2B10 SC-Islet Cells

WT and GAP-2B10 SC-islet cell clusters (5×10⁶ cells) were transplanted under the kidney capsule of B6/albino mice (n=3/group) and graft survival was monitored weekly for nine weeks by bioluminescence following i.p. injection of D-luciferin as described above. At five weeks post-transplantation SC-islet grafts were removed for immunohistochemical analysis of surviving INS+ cells and CD8+ T and FOXP3+ Treg cells.

### Immunohistochemistry of SC-Islet Grafts

The kidneys of B6/Albino mice transplanted with WT and 2B10 SC-islets were excised, fixed overnight in 4% paraformaldehyde (PFA) at room temperature and embedded in paraffin. Sections were pre-cleared with Histo-Clear, rehydrated using an ethanol gradient and antigen fixed by incubating in boiling antigen retrieval reagent (10 mM sodium citrate, pH 6.0) for 50 min. Slides were then blocked in 5% donkey serum for 1 h and stained with Guinea pig anti-human Insulin (DAKO, A0564), Rat anti-mouse CD8a (Biolegend, 100702) and Mouse anti-mouse FOXP3 (Biolegend, 320002) overnight at 4° C. The slides were then washed three times, incubated in secondary antibodies goat anti-mouse 594 (Life Technologies, A-11032), goat anti-rat 488 (Life Technologies, A-11006) and goat anti-guinea pig 647 (Life technologies, A-21450) for 2 h at room temperature, washed, mounted in Vectashield with DAPI (Vector Laboratories; H-1200), covered with coverslips and sealed with clear nail polish. Representative regions were imaged using Zeiss.Z2 with Apotome microscope. All primary and secondary antibodies were used at dilution of 1:200 and 1:500 respectively.

### Statistics

All data are presented as means±SD and were analyzed by GraphPad Prism 9 (GraphPad Software). Statistically significant differences were determined either by one-way or two-way ANOVA, with Tukey's and Sidak's post-hoc test for multiple comparisons, and two-tailed t test for pairwise comparisons. p values are indicated in the figures as *p<0.05, **p<0.01, ***p<0.005 and ****p<0.001.

### Results

### Engineering Hypoimmunogenic SC-Islet Cells

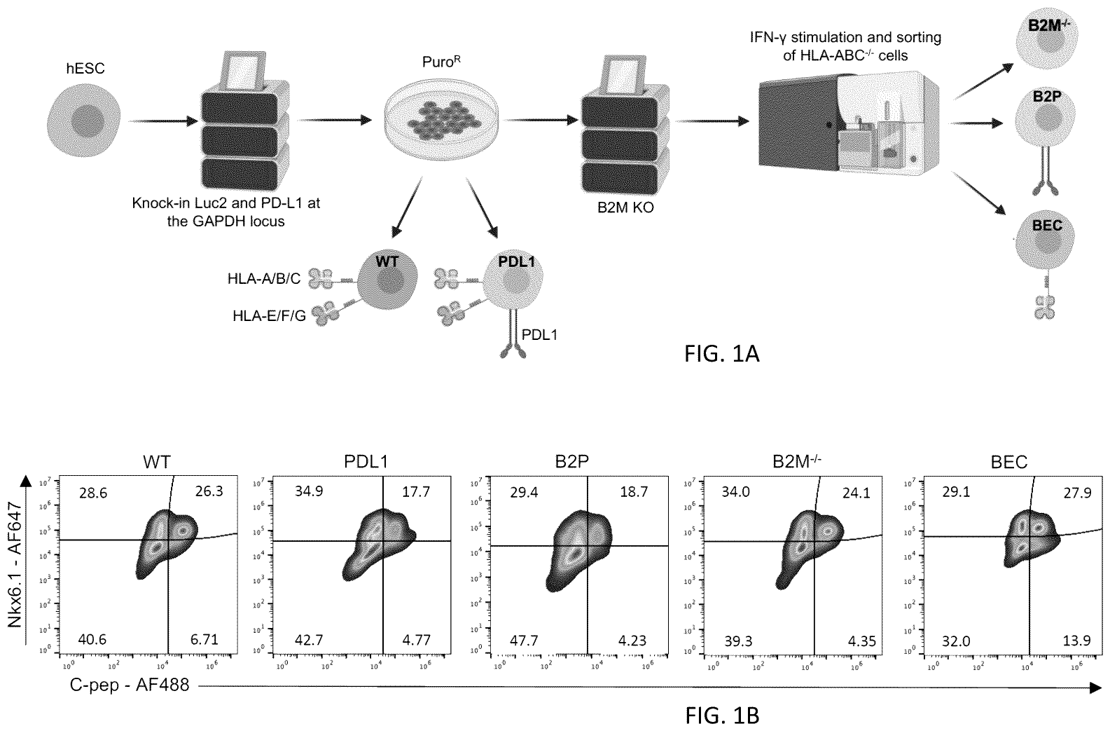

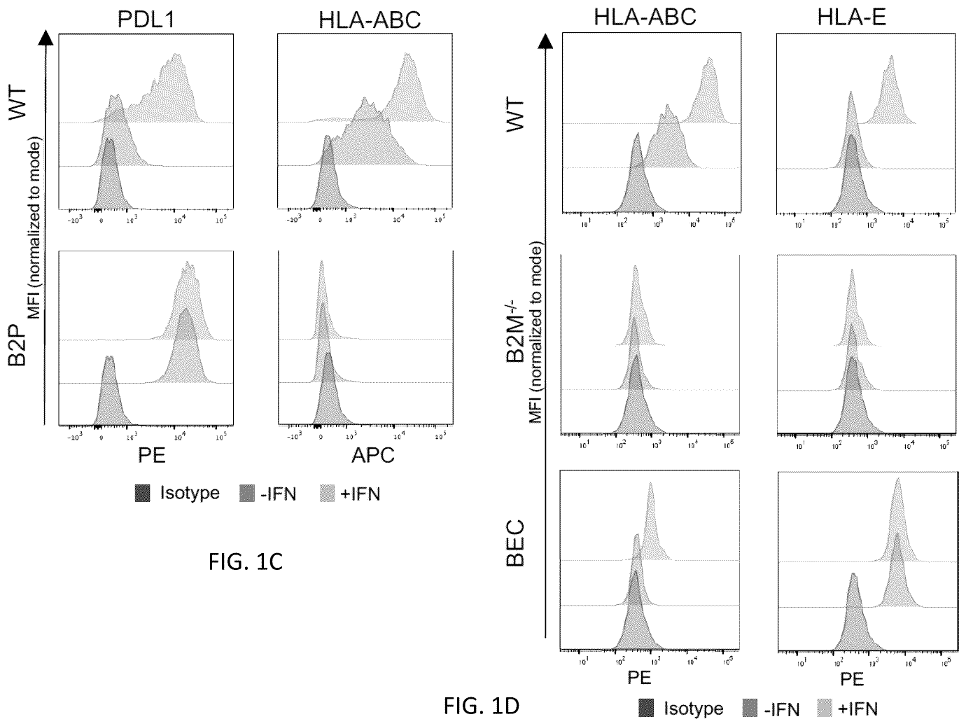

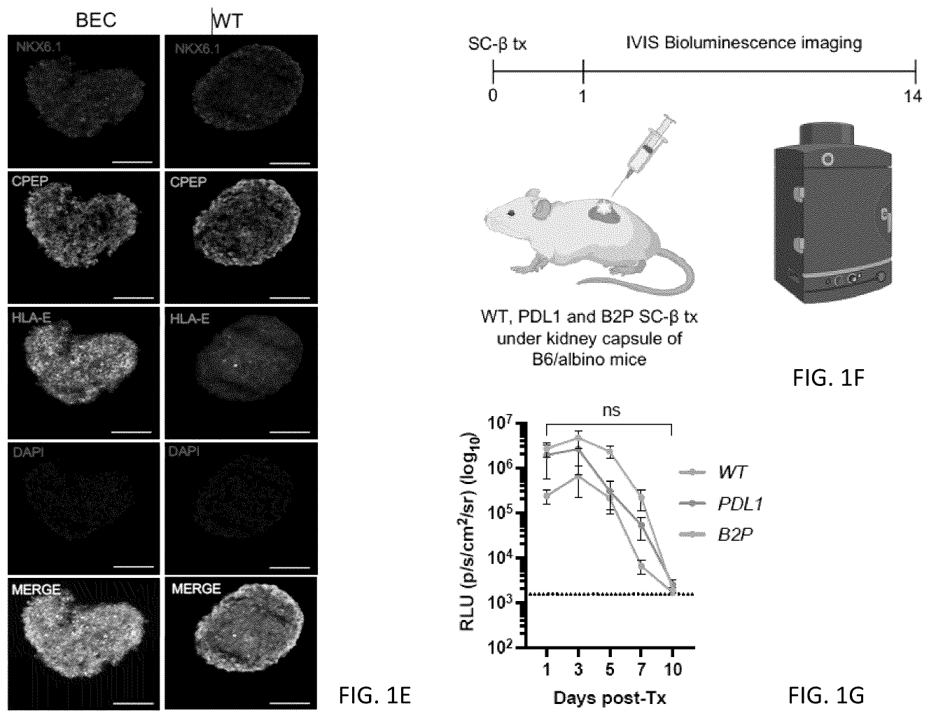

Since GAPDH is constitutively expressed in all cells of the human islet (FIGS. 5A-5B), we targeted the expression of PD-L1 and the HLA-E long-chain fusion to the GAPDH locus of hESCs and used luminescence as a reporter of cell viability. We chose to over-express PD-L1 as it was previously shown to protect SC-islet cells from xeno-rejection (Yoshihara et al., 2020), while the HLA-E long-chain fusion inhibits NK cells in a HLA-deficient context (Gornalusse et al., 2017; Mattapally et al., 2018; Riolobos et al., 2013; Wang et al., 2015). GAPDH-targeting plasmids were modified to include PD-L1 or HLA-E (FIGS. 5C-5E) and used to create five hESCs lines (FIG. 1A)—wild-type (WT), HLA-deficient (B2M−/−), PD-L1-expressing (PD-L1) and HLA-deficient/PD-L1-expressing (B2P) and HLA-deficient/HLA-E-expressing (BEC). All five gene-modified hESC lines successfully differentiated into SC-β cells with similar efficiency as assessed by C-peptide and Nk×6.1 expression (FIG. 1B). Knockout of the beta 2 microglobulin (B2M) gene effectively eliminated HLA class I expression on B2M−/−, B2P and BEC SC-islet cells. After IFN-γ stimulation, HLA class I and PD-L1 were upregulated in WT SC-islet cells (Castro-Gutierrez et al., 2021), whereas B2P SC-islet cells constitutively over-expressed PD-L1 and lacked HLA class I expression (FIG. 1C). A similar expression profile of HLA-E and HLA class I expression was observed on WT and BEC SC-islet cells (FIG. 1D) (Gornalusse et al., 2017). Immunohistochemistry of magnetically-enriched SC-β cells showed localization of the HLA-E long-chain fusion to the membrane of BEC SC-β cells (FIG. 1E) (Veres et al., 2019).

To confirm that human PD-L1 expressed on the surface of SC-islet cells binds PD-1, we assessed binding of fluorescently labelled, soluble human and mouse PD-1-Fc on WT and B2P SC-islet cells (FIG. 5F). Both soluble human and mouse PD-1 bind membrane-bound PD-L1 on B2P SC-islet cells, whereas WT SC-islet cells do not endogenously express sufficient levels of PD-L1 to detectably bind human or mouse PD-1. We also observed a decrease in the binding affinity of soluble mouse PD-1 to human PD-L1 (Freeman et al., 2000). When WT, PD-L1 and B2P SC-islet cells were transplanted under the kidney capsule of B6/albino mice (FIG. 1F), all gene-modified SC-islet cells were rejected within 10 days after transplantation (FIG. 1G). These results suggest that PD-L1 over-expression is not sufficient to protect HUES8-derived SC-islet cells from xeno-rejection, and that ablation of HLA expression does not improve xenograft survival in our animal model of xeno-rejection.

### HLA-Deficient SC-Islet Cells are Resistant to Allogeneic Immune Cell Destruction In Vitro

We next assessed the in vitro survival of gene-modified SC-islet cells in co-culture with allogeneic human immune cells (FIG. 2A). In concordance with previous studies, NK cells and CD4+ and CD8+ T cells represented ˜8, 35 and 10% of enriched PBMCs respectively (FIGS. 6A-6B) (Kleiveland, 2015). When SC-islet cells were pre-treated with IFN-γ and co-cultured with human PBMCs at multiple effector:target ratios, B2M−/− and BEC SC-islet cells demonstrated significantly improved survival in comparison to WT (FIGS. 2B-2C). HLA-E over-expression did not provide additional protective benefit. Additionally, similar survival patterns were observed when SC-islet cells were co-cultured with purified CD8+ and CD4+ T cells (FIGS. 6C-6F). In all co-culture assays, there was no significant difference in the survival of all gene-modified SC-islet cells cultured with CD3/CD28 activated cells.

Since expression of T cell co-activating and co-inhibitory ligands dictates T cell function and is regulated by various stimuli including IFN-γ stimulation (Chen and Flies, 2013), we performed bulk RNA sequencing on WT CD49a+SC-β cells and assessed T cell ligand expression after IFN-γ stimulation (FIG. 2D). As expected, IFN-γ stimulation upregulated the T cell co-inhibitory ligands PD-L1 and LGALS9 (galectin 9) in SC-β cells (FIG. 2E) (Garcia-Diaz et al., 2017; Imaizumi et al., 2002). Conversely, while we did not detect transcripts for many T cell co-activating ligands other than HLA genes, we found that IFN-γ stimulation upregulated members of the TNF receptor superfamily HVEM (TNF Receptor Superfamily Member 14) and CD40 (Tumor Necrosis Factor Receptor Superfamily Member 5) in SC-β cells (FIG. 2F) (Benci et al., 2016; Wagner et al., 2002). Interestingly, BNT3A1 (butyrophilin subfamily 3 member A1), which is an MHC-associated gene that regulates T cell activation and proliferation (Rigau et al., 2020), was also upregulated. These results suggest that in the absence of classical HLA-TCR signaling due to HLA knockout, other co-stimulatory and co-inhibitory T cell ligands are expressed in SC-β cells that may influence T cell function.

### HLA-Deficient SC-Islet Cells are Resistant to Allogeneic NK Cell Destruction In Vitro and In Vivo

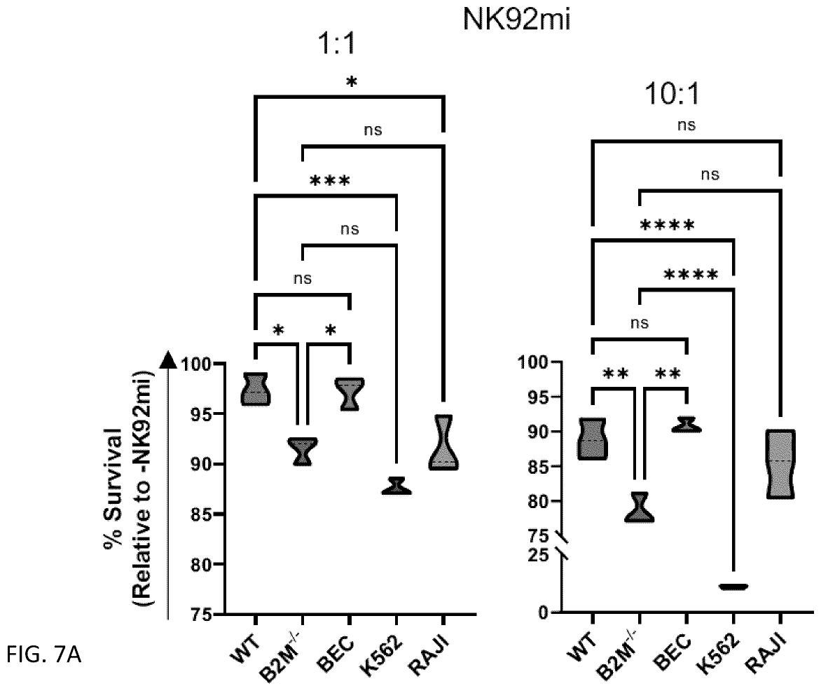

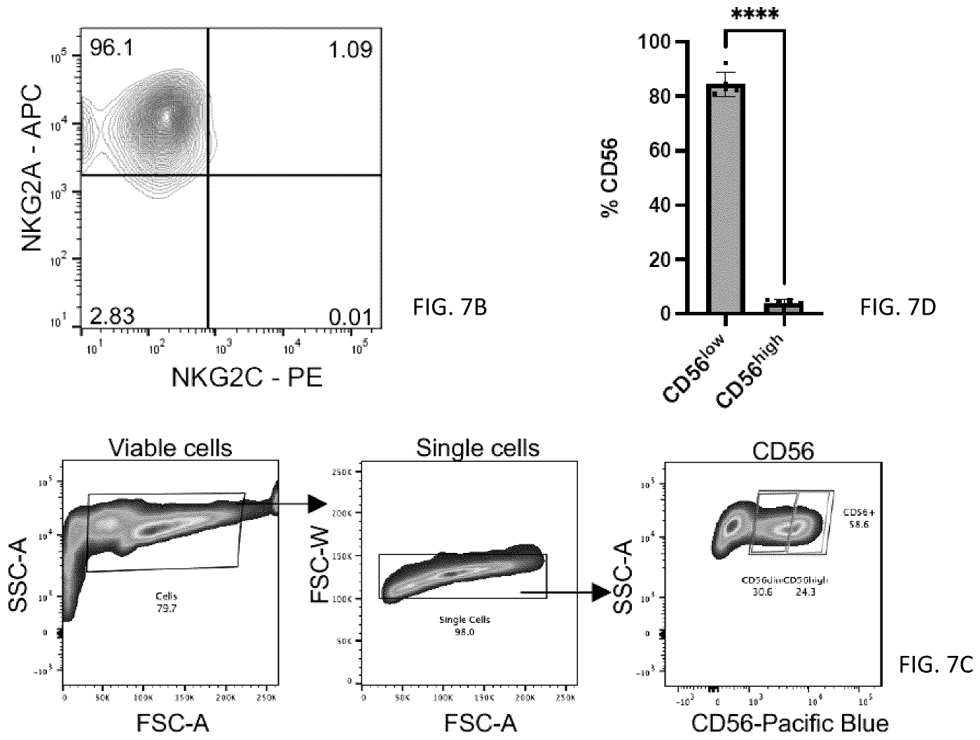

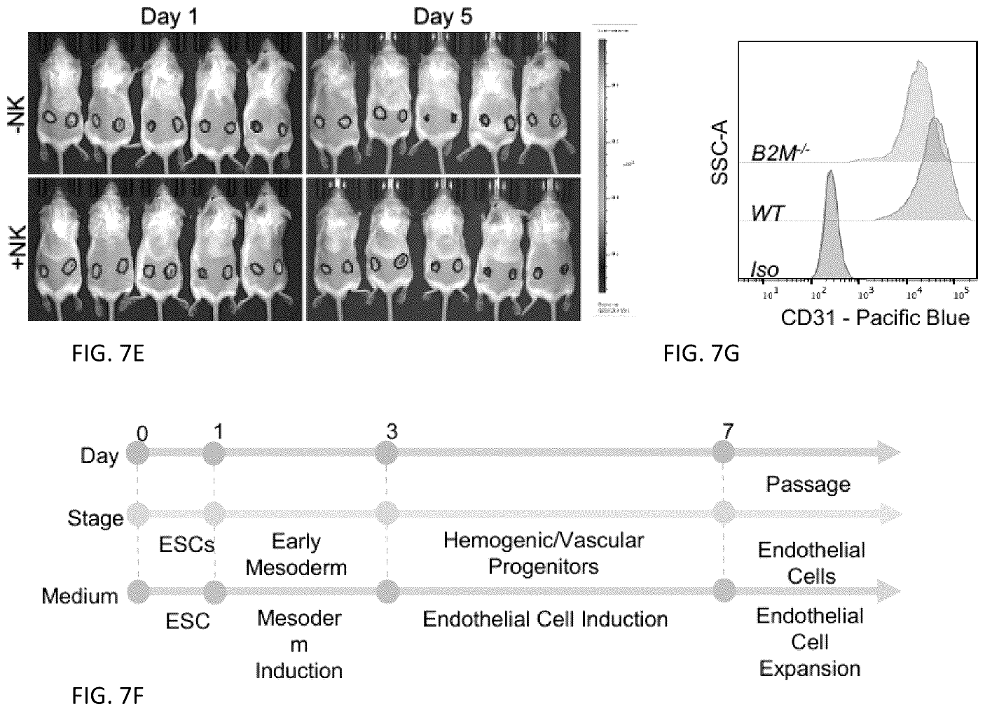

We next assessed the effect of HLA deletion and HLA-E over-expression on NK cell function against SC-islet cells. When co-cultured with NK92mi cells, BEC SC-islet cells showed no significant difference in survival in comparison to WT SC-islet cells, whereas HLA-deficient SC-islet cells were susceptible to NK92mi cytotoxicity (FIG. 7A) (Gornalusse et al., 2017). The strong protective effect of HLA-E against NK92mi cells is likely due to the high percentage (˜96.1%) of NKG2A+/NKG2C− cells (FIG. 7B), which biases NK cell inhibition.

While NK cell lines are useful tools for assessing NK cell cytotoxicity, they do not accurately recapitulate primary NK cell receptor expression and function. Previous studies have shown that in the absence of IL-2 activation, human NK cells do not destroy HLA-deficient endothelial cells and platelets (Deuse et al., 2021; Suzuki et al., 2020). Thus, prior to co-culture with gene-modified SC-islet cells, we pre-activated human NK cells with IL-2 for 5 days as previously described (Deuse et al., 2021). Enriched NK cells consisted of ˜80% CD56dim and ˜5% CD56high NK cells (FIGS. 7C-7D). Surprisingly, despite IL-2 pre-activation, we observed no significant difference in survival between gene-modified SC-islet cells at multiple effector:target ratios (FIG. 2G). Since HLA-E over-expression does not provide any additional protective benefit to HLA-deficient SC-islet cells against NK cell cytotoxicity, we chose to interogate WT and B2M−/− SC-islet cells moving forward. We assessed the survival of WT and B2M−/− SC-islet cells after transplantation with IL-2 pre-activated NK cells in Scid/beige mice (FIG. 2H). Again, there was no significant difference in WT and B2M−/− SC-islet cell survival in vivo (FIG. 2I, FIG. 7E). Taken together, these results suggest that HLA-deficient SC-islet cells are intrinsically resistant to NK cell cytotoxicity and that their survival in vitro is recapitulated in vivo.

Exclusively, SC-β Cells Intrinsically Possess and Sustain an NK Cell Evasive Ligand Profile after Inflammatory Stimulus

Since NK cell function is dictated by a balance of inhibitory and activating signals, we compared the ligand profile of CD49a+SC-β cells to stem cell-derived endothelial (SC-endo) cells. We chose endothelial cells as a comparative cell type because it has previously been shown that HLA-deficient endothelial cells are susceptible to pre-activated NK cells (Deuse et al., 2021). Endothelial cell differentiation resulted in >95% CD31+SC-Endo cells derived from both WT and B2M−/− hESCs (FIGS. 7F-7G). Bulk RNA sequencing and transcript analysis of NK cell ligands in SC-β and SC-endo cells confirmed that SC-endo cells possess an activating NK cell ligand profile, characterized by expression of MICA, MICB, ULBP1, ULBP2 and RAETIG, whereas transcripts for these genes could not be detected in SC-β cells (FIGS. 3A-3B). Importantly, we found that the expression of most NK cell ligands is not IFN-γ regulated, except for the HLA molecules. Thus, within an HLA-deficient context, the expression of non-HLA NK cell ligands is stable following an inflammatory stimulus. We confirmed the bulk RNA sequencing analysis of NK cell ligand expression by flow cytometry, which showed that MIC and ULBP proteins were either absent or less expressed on SC-β cells (FIGS. 3C-3D). Taken together these data suggest that SC-β cells intrinsically possess and sustain a diminished NK cell activating ligand profile after inflammatory stimulus, and that this ligand phenotype may explain their resistance to IL-2 pre-activated NK cell cytotoxicity.

### HLA-Deficient SC-Islet Cells Reverse Diabetes in Humanized Mice and Demonstrate Delayed Graft Rejection

Having demonstrated that HLA-deficient SC-islet cells resist PBMC cytotoxicity in vitro and NK cell cytotoxicity in vitro and in vivo, we assessed the survival of HLA-deficient SC-islet cells in diabetic, NSG-DKO mice prior to PBMC injection (FIG. 3E). By 10 weeks post-transplantation there was no significant difference in blood glucose levels of mice transplanted with WT and B2M−/− SC-islet cells (FIG. 3F). Once blood glucose levels had been normalized by the SC-islet transplants, we injected HLA-A2-(mis-matched) PBMCs and assessed allograft rejection by monitoring blood glucose levels. WT SC-islet cells were destroyed within 2 weeks, whereas the rejection of B2M−/− SC-islet cells was delayed. At 7 weeks post-PBMC injection, in vivo GSIS assay showed that animals transplanted with WT SC-islet cells lost in vivo graft function, while some graft function was observed in animals transplanted with B2M−/− SC-islet cells (FIG. 3G). These results suggest that B2M−/− SC-islet cells reverse diabetes and demonstrate delayed allograft rejection.

Cytokine-Secreting SC-β Cells Induce a Local Tolerogenic Microenvironment that Protects Against Xeno-Rejection

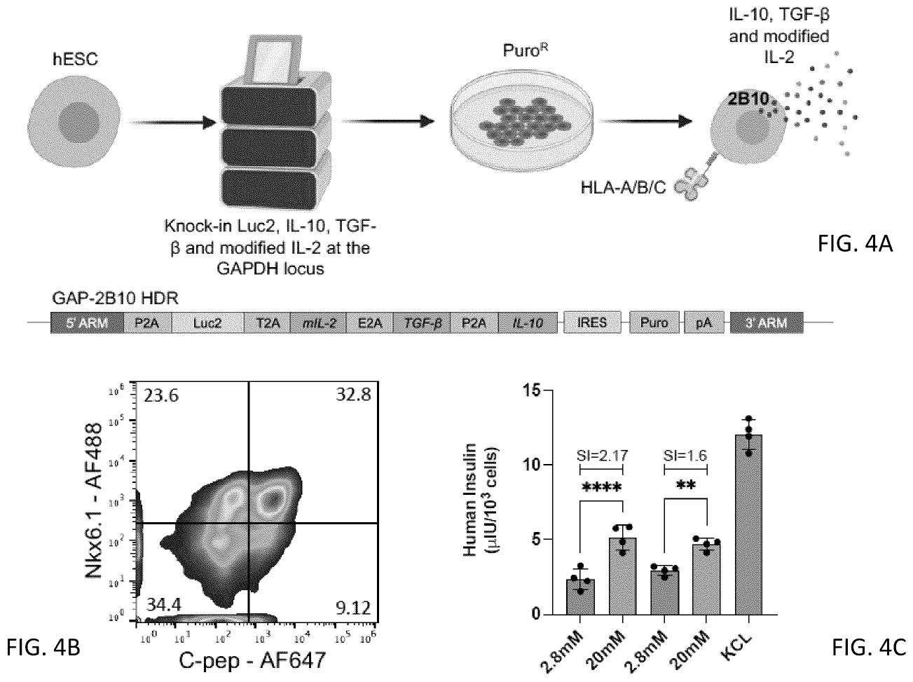

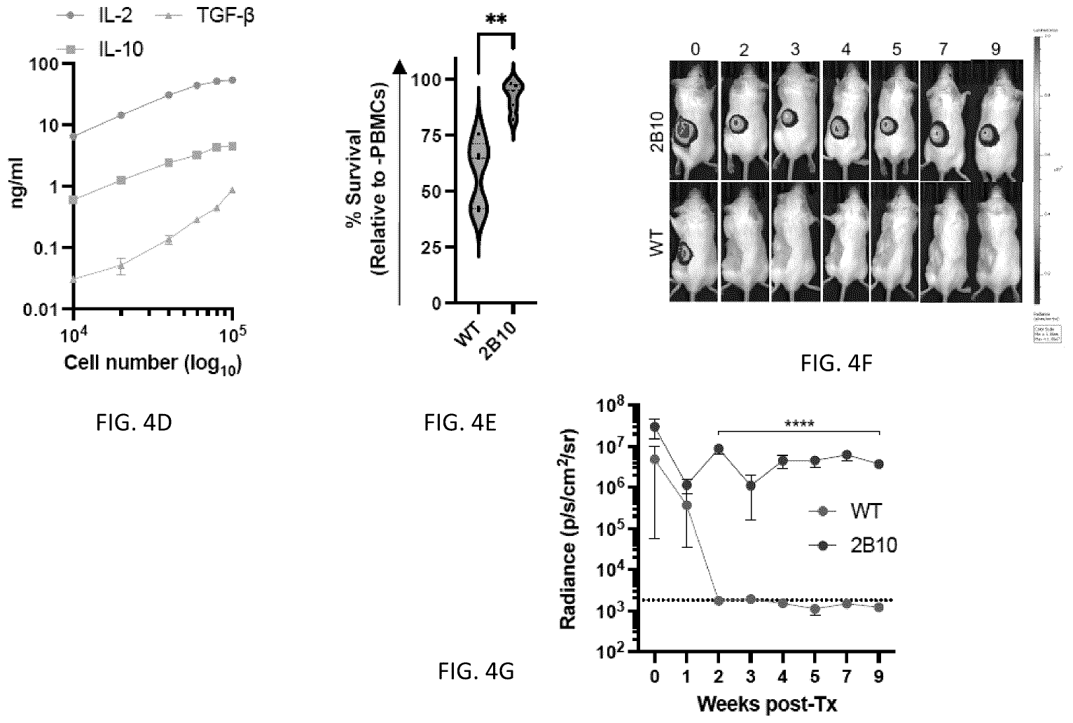

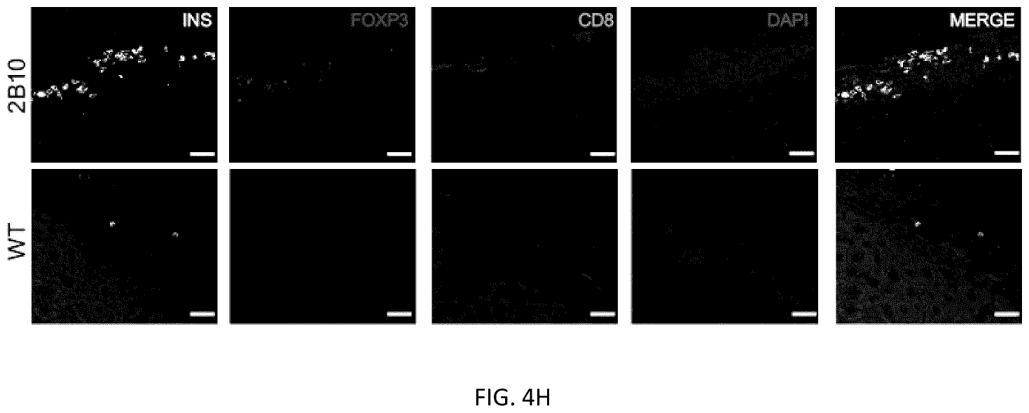

As a result of the varying success of immune-evasive engineering to protect SC-islet cells, we pursued a complementary strategy by engineering SC-islet cells to secrete the cytokines IL-2 mutein, TGF-β and IL-10 as vehicles of localized immune (FIG. 4A). The IL-2 mutein (N88D) possesses reduced affinity for the IL-2RO7 receptor, resulting in the production of a Treg-selective molecule that preferentially expands Tregs while having a minimal effect on CD4+ and CD8+ memory T cells (Khoryati et al., 2020; Peterson et al., 2018). Since the immune-suppressive phenotype of Tregs requires IL-10 and TGF-β, we included these two cytokines in our immune-tolerizing construct (Horwitz et al., 2008).

Differentiation of this genetically-modified cell line, called 2B10, resulted in ˜30% Nk×6.1+/C-peptide+SC-β cells (FIG. 4B) with physiological function as assessed in vitro by GSIS (FIG. 4C). We also confirmed that 2B10 SC-islet cells secrete modified IL-2, TGF-β and IL-10, and that the quantity was cell-concentration dependent (FIG. 4D). When WT and 2B10 SC-islet cells were co-cultured with human PBMCs in vitro, 2B10 SC-islet cells showed significantly improved survival (FIG. 4E). Notably, 2B10 SC-islet cells transplanted under the kidney capsule of B6/albino mice survived up to 9 weeks post-transplantation, while WT SC-islet cells were destroyed within 2 weeks (FIGS. 4F-4G). Furthermore, we found that WT grafts contained little to no remaining SC-islet cells, while 2B10 grafts contained surviving insulin-producing cells and Tregs localized within the graft (FIG. 4H). Collectively, these data suggest that SC-islet cells can be engineered to co-secrete immune-modulatory cytokines that induce a tolerogenic local microenvironment characterized by Treg cell infiltration that sustains xenograft survival.

### REFERENCES

Abou-Daya, K. I., Tieu, R., Zhao, D., Rammal, R., Sacirbegovic, F., Williams, A. L., Shlomchik, W. D., Oberbarnscheidt, M. H., and Lakkis, F. G. (2021). Resident memory T cells form during persistent antigen exposure leading to allograft rejection. Science Immunology 6, eabc8122.
Alagpulinsa, D. A., Cao, J. J. L., Driscoll, R. K., Sîrbulescu, R. F., Penson, M. F. E., Sremac, M., Engquist, E. N., Brauns, T. A., Markmann, J. F., Melton, D. A., et al. (2019). Alginate-microencapsulation of human stem cell-derived β cells with CXCL12 prolongs their survival and function in immunocompetent mice without systemic immunosuppression. Am J Transplant 19, 1930-1940.
Atkinson, M. A., and Maclaren, N. K. (1994). The Pathogenesis of Insulin-Dependent Diabetes Mellitus. The New England Journal of Medicine 19, 1428-1436.
Benci, J. L., Xu, B., Qiu, Y., Wu, T. J., Dada, H., Twyman-Saint Victor, C., Cucolo, L., Lee, D. S. M., Pauken, K. E., Huang, A. C., et al. (2016). Tumor Interferon Signaling Regulates a Multigenic Resistance Program to Immune Checkpoint Blockade. Cell 167, 1540-1554.
Bochenek, M. A., Veiseh, O., Vegas, A. J., McGarrigle, J. J., Qi, M., Marchese, E., Omami, M., Doloff, J. C., Mendoza-Elias, J., Nourmohammadzadeh, M., et al. (2018). Alginate encapsulation as long-term immune protection of allogeneic pancreatic islet cells transplanted into the omental bursa of macaques. Nat Biomed Eng 2, 810-821.
Castro-Gutierrez, R., Alkanani, A., Mathews, C. E., Michels, A., and Russ, H. A. (2021). Protecting Stem Cell Derived Pancreatic Beta-Like Cells From Diabetogenic T Cell Recognition. Frontiers in Endocrinology 12.
Chen, L., and Flies, D. B. (2013). Molecular mechanisms of T cell co-stimulation and co-inhibition. Nat Rev Immunol 13, 227-242.
D'Amour, K. A., Bang, A. G., Eliazer, S., Kelly, O. G., Agulnick, A. D., Smart, N. G., Moorman, M. A., Kroon, E., Carpenter, M. K., and Baetge, E. E. (2006). Production of pancreatic hormone-expressing endocrine cells from human embryonic stem cells. Nat Biotechnol 24, 1392-1401.
Deuse, T., Hu, X., Agbor-Enoh, S., Jang, M. K., Alawi, M., Saygi, C., Gravina, A., Tediashvili, G., Nguyen, V. Q., Liu, Y., et al. (2021). The SIRPα-CD47 immune checkpoint in NK cells. The Journal of experimental medicine 218.
Deuse, T., Hu, X., Gravina, A., Wang, D., Tediashvili, G., De, C., Thayer, W. O., Wahl, A., Garcia, J. V., Reichenspurner, H., et al. (2019). Hypoimmunogenic derivatives of induced pluripotent stem cells evade immune rejection in fully immunocompetent allogeneic recipients. Nature Biotechnology 37, 252-258.
Finger, L. R., Pu, J., Wasserman, R., Vibhakar, R., Louie, E., Hardy, R. R., Burrows, P. D., and Billips, L. G. (1997). The human PD-1 gene: complete cDNA, genomic organization, and developmentally regulated expression in B cell progenitors. Gene 197, 177-187.
Freeman, G. J., Long, A. J., Iwai, Y., Bourque, K., Chernova, T., Nishimura, H., Fitz, L. J., Malenkovich, N., Okazaki, T., Byrne, M. C., et al. (2000). Engagement of the PD-1 immunoinhibitory receptor by a novel B7 family member leads to negative regulation of lymphocyte activation. The Journal of experimental medicine 192, 1027-1034.
Furman, B. L. (2021). Streptozotocin-Induced Diabetic Models in Mice and Rats. Current Protocols 1, e78.
Garcia-Diaz, A., Shin, D. S., Moreno, B. H., Saco, J., Escuin-Ordinas, H., Rodriguez, G. A., Zaretsky, J. M., Sun, L., Hugo, W., Wang, X., et al. (2017). Interferon Receptor Signaling Pathways Regulating PD-L1 and PD-L2 Expression. Cell reports 19, 1189-1201.
Gerace, D., Boulanger, K. R., Hyoje-Ryu Kenty, J., and Melton, D. A. (2021). Generation of a heterozygous GAPDH-Luciferase human ESC line (HVRDe008-A-1) for in vivo monitoring of stem cells and their differentiated progeny. Stem Cell Research 53, 102371.
Gornalusse, G. G., Hirata, R. K., Funk, S. E., Riolobos, L., Lopes, V. S., Manske, G., Prunkard, D., Colunga, A. G., Hanafi, L.-A., Clegg, D. O., et al. (2017). HLA-E-expressing pluripotent stem cells escape allogeneic responses and lysis by NK cells. Nature Biotechnology 35, 765.
Han, X., Wang, M., Duan, S., Franco, P. J., Kenty, J. H.-R., Hedrick, P., Xia, Y., Allen, A., Ferreira, L. M. R., Strominger, J. L., et al. (2019). Generation of hypoimmunogenic human pluripotent stem cells. Proceedings of the National Academy of Sciences 116, 10441.
Harding, J., Vintersten-Nagy, K., Shutova, M., Yang, H., Tang, J. K., Massumi, M., Izaidfar, M., Izadifar, Z., Zhang, P., Li, C., et al. (2019). Induction of long-term allogeneic cell acceptance and formation of immune privileged tissue in immunocompetent hosts. bioRxiv, 716571.
Hartemann, A., Bensimon, G., Payan, C. A., Jacqueminet, S., Bourron, O., Nicolas, N., Fonfrede, M., Rosenzwajg, M., Bernard, C., and Klatzmann, D. (2013). Low-dose interleukin 2 in patients with type 1 diabetes: a phase 1/2 randomised, double-blind, placebo-controlled trial. Lancet Diabetes Endocrinol 1, 295-305.
Herbst, F., Ball, C. R., Tuorto, F., Nowrouzi, A., Wang, W., Zavidij, O., Dieter, S. M., Fessler, S., van der Hoeven, F., Kloz, U., et al. (2012). Extensive Methylation of Promoter Sequences Silences Lentiviral Transgene Expression During Stem Cell Differentiation In Vivo. Molecular Therapy 20, 1014-1021.
Herndler-Brandstetter, D., Shan, L., Yao, Y., Stecher, C., Plajer, V., Lietzenmayer, M., Strowig, T., de Zoete, M. R., Palm, N. W., Chen, J., et al. (2017). Humanized mouse model supports development, function, and tissue residency of human natural killer cells. Proceedings of the National Academy of Sciences 114, E9626.
Herold, K. C., Bundy, B. N., Long, S. A., Bluestone, J. A., DiMeglio, L. A., Dufort, M. J., Gitelman, S. E., Gottlieb, P. A., Krischer, J. P., Linsley, P. S., et al. (2019). An Anti-CD3 Antibody, Teplizumab, in Relatives at Risk for Type 1 Diabetes. New England Journal of Medicine 381, 603-613.
Heusschen, R., Griffioen, A. W., and Thijssen, V. L. (2013). Galectin-9 in tumor biology: A jack of multiple trades. Biochimica et Biophysica Acta (BBA)—Reviews on Cancer 1836, 177-185.
Horwitz, D. A., Zheng, S. G., Wang, J., and Gray, J. D. (2008). Critical role of IL-2 and TGF-beta in generation, function and stabilization of Foxp3+CD4+ Treg. Eur J Immunol 38, 912-915.
Imaizumi, T., Kumagai, M., Sasaki, N., Kurotaki, H., Mori, F., Seki, M., Nishi, N., Fujimoto, K., Tanji, K., Shibata, T., et al. (2002). Interferon-gamma stimulates the expression of galectin-9 in cultured human endothelial cells. J Leukoc Biol 72, 486-491.
Kaiser, B. K., Barahmand-pour, F., Paulsene, W., Medley, S., Geraghty, D. E., and Strong, R. K. (2005). Interactions between NKG2× Immunoreceptors and HLA-E Ligands Display Overlapping Affinities and Thermodynamics. The Journal of Immunology 174, 2878.
Khoryati, L., Pham, M. N., Sherve, M., Kumari, S., Cook, K., Pearson, J., Bogdani, M., Campbell, D. J., and Gavin, M. A. (2020). An IL-2 mutein engineered to promote expansion of regulatory T cells arrests ongoing autoimmunity in mice. Science immunology 5, eaba5264.
Kleiveland, C. R. (2015). Peripheral Blood Mononuclear Cells. In The Impact of Food Bioactives on Health: in vitro and ex vivo models, K. Verhoeckx, P. Cotter, I. López-Expósito, C. Kleiveland, T. Lea, A. Mackie, T. Requena, D. Swiatecka, and H. Wichers, eds. (Cham: Springer International Publishing), pp. 161-167.
Leite, N. C., Pelayo, G. C., and Melton, D. A. (2022). Genetic manipulation of stress pathways can protect stem-cell-derived islets from apoptosis in vitro. Stem cell reports.
Lim, D., Sreekanth, V., Cox, K. J., Law, B. K., Wagner, B. K., Karp, J. M., and Choudhary, A. (2020). Engineering designer beta cells with a CRISPR-Cas9 conjugation platform. Nature communications 11, 4043.
Mandal, P. K., Ferreira, L. M., Collins, R., Meissner, T. B., Boutwell, C. L., Friesen, M., Vrbanac, V., Garrison, B. S., Stortchevoi, A., Bryder, D., et al. (2014). Efficient ablation of genes in human hematopoietic stem and effector cells using CRISPR/Cas9. Cell stem cell 15, 643-652.
Mattapally, S., Pawlik, K. M., Fast, V. G., Zumaquero, E., Lund, F. E., Randall, T. D., Townes, T. M., and Zhang, J. (2018). Human Leukocyte Antigen Class I and II Knockout Human Induced Pluripotent Stem Cell-Derived Cells: Universal Donor for Cell Therapy. J Am Heart Assoc 7, e010239.
Millman, J. R., Xie, C., Van Dervort, A., Gurtler, M., Pagliuca, F. W., and Melton, D. A. (2016). Generation of stem cell-derived beta-cells from patients with type 1 diabetes. Nature communications 7, 11463.
Monti, P., Scirpoli, M., Maffi, P., Ghidoli, N., De Taddeo, F., Bertuzzi, F., Piemonti, L., Falcone, M., Secchi, A., and Bonifacio, E. (2008). Islet transplantation in patients with autoimmune diabetes induces homeostatic cytokines that expand autoreactive memory T cells. The Journal of clinical investigation 118, 1806-1814.
Nair, G. G., Liu, J. S., Russ, H. A., Tran, S., Saxton, M. S., Chen, R., Juang, C., Li, M. L., Nguyen, V. Q., Giacometti, S., et al. (2019). Recapitulating endocrine cell clustering in culture promotes maturation of human stem-cell-derived beta cells. Nature cell biology 21, 263-274.
Oberbarnscheidt, M. H., Zeng, Q., Li, Q., Dai, H., Williams, A. L., Shlomchik, W. D., Rothstein, D. M., and Lakkis, F. G. (2014). Non-self recognition by monocytes initiates allograft rejection. The Journal of clinical investigation 124, 3579-3589.
Pagliuca, F. W., Millman, J. R., Gurtler, M., Segel, M., Van Dervort, A., Ryu, J. H., Peterson, Q. P., Greiner, D., and Melton, D. A. (2014). Generation of functional human pancreatic beta cells in vitro. Cell 159, 428-439.
Parent, A. V., Faleo, G., Chavez, J., Saxton, M., Berrios, D. I., Kerper, N. R., Tang, Q., and Hebrok, M. (2021). Selective deletion of human leukocyte antigens protects stem cell-derived islets from immune rejection. Cell Reports 36, 109538.
Peterson, L. B., Bell, C. J. M., Howlett, S. K., Pekalski, M. L., Brady, K., Hinton, H., Sauter, D., Todd, J. A., Umana, P., Ast, O., et al. (2018). A long-lived IL-2 mutein that selectively activates and expands regulatory T cells as a therapy for autoimmune disease. Journal of Autoimmunity 95, 1-14.
Raudvere, U., Kolberg, L., Kuzmin, I., Arak, T., Adler, P., Peterson, H., and Vilo, J. (2019). g:Profiler: a web server for functional enrichment analysis and conversions of gene lists (2019 update). Nucleic acids research 47, W191-W198.
Rezania, A., Bruin, J. E., Arora, P., Rubin, A., Batushansky, I., Asadi, A., O'Dwyer, S., Quiskamp, N., Mojibian, M., Albrecht, T., et al. (2014). Reversal of diabetes with insulin-producing cells derived in vitro from human pluripotent stem cells. Nat Biotechnol 32, 1121-1133.
Rigau, M., Ostrouska, S., Fulford Thomas, S., Johnson Darryl, N., Woods, K., Ruan, Z., McWilliam Hamish, E. G., Hudson, C., Tutuka, C., Wheatley Adam, K., et al. (2020). Butyrophilin 2A1 is essential for phosphoantigen reactivity by γδ T cells. Science 367, eaay5516.
Riolobos, L., Hirata, R. K., Turtle, C. J., Wang, P. R., Gornalusse, G. G., Zavajlevski, M., Riddell, S. R., and Russell, D. W. (2013). HLA engineering of human pluripotent stem cells. Molecular therapy: the journal of the American Society of Gene Therapy 21, 1232-1241.
Russ, H. A., Parent, A. V., Ringler, J. J., Hennings, T. G., Nair, G. G., Shveygert, M., Guo, T., Puri, S., Haataja, L., Cirulli, V., et al. (2015). Controlled induction of human pancreatic progenitors produces functional beta-like cells in vitro. The EMBO journal 34, 1759-1772.
Shapiro, A. M., Ricordi, C., Hering, B. J., Auchincloss, H., Lindblad, R., Robertson, R. P., Secchi, A., Brendel, M. D., Berney, T., Brennan, D. C., et al. (2006). International trial of the Edmonton protocol for islet transplantation. N Engl J Med 355, 1318-1330.
Sintov, E., Gerace, D., and Melton, D. A. (2021). A human ESC line for efficient CRISPR editing of pluripotent stem cells. Stem Cell Research 57, 102591.
Stegall, M. D., Lafferty, K. J., Kam, I., and Gill, R. G. (1996). Evidence of recurrent autoimmunity in human allogeneic islet transplantation. Transplantation 61, 1272-1274.
Suzuki, D., Flahou, C., Yoshikawa, N., Stirblyte, I., Hayashi, Y., Sawaguchi, A., Akasaka, M., Nakamura, S., Higashi, N., Xu, H., et al. (2020). iPSC-Derived Platelets Depleted of HLA Class I Are Inert to Anti-HLA Class I and Natural Killer Cell Immunity. Stem cell reports 14, 49-59.
Veres, A., Faust, A. L., Bushnell, H. L., Engquist, E. N., Kenty, J. H., Harb, G., Poh, Y. C., Sintov, E., Gurtler, M., Pagliuca, F. W., et al. (2019). Charting cellular identity during human in vitro beta-cell differentiation. Nature 569, 368-373.
Viricel, C., Ahmed, M., and Barakat, K. (2015). Human PD-1 binds differently to its human ligands: a comprehensive modeling study. J Mol Graph Model 57, 131-142.
Wagner, A. H., Gebauer, M., Pollok-Kopp, B., and Hecker, M. (2002). Cytokine-inducible CD40 expression in human endothelial cells is mediated by interferon regulatory factor-1. Blood 99, 520-525.
Wang, D., Quan, Y., Yan, Q., Morales, J. E., and Wetsel, R. A. (2015). Targeted Disruption of the 02-Microglobulin Gene Minimizes the Immunogenicity of Human Embryonic Stem Cells. Stem cells translational medicine 4, 1234-1245.
Wen, J., Wu, J., Cao, T., Zhi, S., Chen, Y., Aagaard, L., Zhen, P., Huang, Y., Zhong, J., and Huang, J. (2021). Methylation silencing and reactivation of exogenous genes in lentivirus-mediated transgenic mice. Transgenic Res 30, 63-76.
Wyburn, K. R., Jose, M. D., Wu, H., Atkins, R. C., and Chadban, S. J. (2005). The Role of Macrophages in Allograft Rejection. Transplantation 80.
Xu, H., Wang, B., Ono, M., Kagita, A., Fujii, K., Sasakawa, N., Ueda, T., Gee, P., Nishikawa, M., Nomura, M., et al. (2019). Targeted Disruption of HLA Genes via CRISPR-Cas9 Generates iPSCs with Enhanced Immune Compatibility. Cell stem cell 24, 566-578.e567.
Yang, R., Sun, L., Li, C.-F., Wang, Y.-H., Yao, J., Li, H., Yan, M., Chang, W.-C., Hsu, J.-M., Cha, J.-H., et al. (2021). Galectin-9 interacts with PD-1 and TIM-3 to regulate T cell death and is a target for cancer immunotherapy. Nature communications 12, 832.
Yoshihara, E., O'Connor, C., Gasser, E., Wei, Z., Oh, T. G., Tseng, T. W., Wang, D., Cayabyab, F., Dai, Y., Yu, R. T., et al. (2020). Immune-evasive human islet-like organoids ameliorate diabetes. Nature 586, 606-611.
Zhuang, Q., Liu, Q., Divito, S. J., Zeng, Q., Yatim, K. M., Hughes, A. D., Rojas-Canales, D. M., Nakao, A., Shufesky, W. J., Williams, A. L., et al. (2016). Graft-infiltrating host dendritic cells play a key role in organ transplant rejection. Nature communications 7, 12623-12623.

1. A hypoimmunogenic cell engineered to express one or more tolerogenic transgenes from one or more loci in the stem cell or progenitor cell which is not silenced during differentiation of the stem cell or progenitor cell to a desired differentiated cell type.

2.-16. (canceled)

17. A method of producing a hypoimmunogenic cell comprising engineering a stem cell or progenitor cell to express one or more tolerogenic transgenes from a locus in the stem cell or progenitor cell which is not silenced during differentiation of the cell to a desired cell type and exposing the stem cell or progenitor cell to conditions suitable for the stem cell or progenitor cell to differentiate to a desired cell type.

18.-32. (canceled)

33. A method of mediating localized immune tolerance comprising engineering a stem cell or progenitor cell to express one or more immunomodulatory agents from a locus in the stem cell or progenitor cell which is not silenced during differentiation of the cell to a desired cell type and exposing the stem cell or progenitor cell to conditions suitable for the stem cell or progenitor cell to differentiate to a desired cell type, thereby producing a desired cell type that secretes said one or more immunomodulatory agents.

34.-45. (canceled)

46. A cell engineered to express one or more immunomodulatory agents from one or more loci in a stem cell or progenitor cell which is not silenced during differentiation of the stem cell or progenitor cell to a desired differentiated cell type, wherein the one or more immunomodulatory agents is selected from the group consisting of IL-10, TGF-β and IL-2 or a modified IL-2.

47. A cell according to claim 46, wherein the cell is a stem cell.

48. A cell according to claim 46, wherein the cell is an induced pluripotent cell.

49. A cell according to claim 46, wherein the cell is a progenitor cell.

50. A cell according to claim 46, wherein the cell is a beta cell progenitor cell.

51. A cell according to claim 46, wherein the cell is a differentiated cell.

52. A cell according to claim 46, wherein the cell is a beta cell.

53. A cell according to claim 46, wherein the cell is an alpha cell.

54. A cell according to claim 46, wherein the cell is an islet cell.

55. A cell according to claim 46, wherein the one or more loci is the locus of a housekeeping gene.

56. A cell according to claim 55, wherein the housekeeping gene is selected from the group consisting of actin, ubiquitin, and GAPDH.

57. (canceled)

58. (canceled)

59. A cell produced by the method of claim 17.

60. A cell produced by the method of claim 33.

61. A method of treating a subject in need thereof comprising administering to the subject a cell according to claim 1.

62. A method of treating a subject in need thereof comprising administering to the subject a cell according to claim 46.
